(19) United States
(12) Patent Application Publication
(10) Pub. No.: US 2017/0216439 A1
(43) Pub. Date: Aug. 3, 2017

(54) OTIC FORMULATIONS FOR THE TREATMENT OF CERUMINOSIS

(71) Applicants: Otonomy, Inc., San Diego, CA (US); RosENTbloom LP, San Antonio, TX (US)

(72) Inventors: Carl Lebel, Malibu, CA (US); Jeffrey S. Rosenbloom, San Antonio, TX (US)

(21) Appl. No.: 15/329,161

(22) PCT Filed: Jul. 29, 2015
(86) PCT No.: PCT/US2015/042634

§ 371 Date: Jan. 25, 2017

Related U.S. Application Data
(60) Provisional application No. 62/030,487, filed on Jul. 29, 2014.

(51) Int. Cl. A61K 47/10; A61K 31/221; A61K 31/27; A61K 31/4178; A61K 31/407; A61K 31/4439; A61K 31/519; A61K 31/496; A61K 31/4168; A61K 31/138; A61K 31/165; A61K 31/517; A61K 31/137; A61K 31/01; A61K 31/565; A61K 31/573; A61K 9/00; A61K 38/00; A61K 9/06

(52) U.S. Cl. CPC A61K 47/10; A61K 9/06; A61K 31/221; A61K 31/27; A61K 31/4178; A61K 31/407; A61K 31/4439; A61K 31/519; A61K 31/496; A61K 31/4168; A61K 31/138; A61K 31/165; A61K 31/517; A61K 31/137; A61K 31/01; A61K 31/565; A61K 31/573; A61K 9/0046; A61K 38/00

(57) ABSTRACT

Disclosed herein are compositions, formulations, methods, devices and kits for modulating the production of cerumen, the treatment of ceruminosis, and the treatment of ceruminosis associated diseases or conditions. In these methods, the otic compositions and formulations are administered locally to an individual afflicted with ceruminosis and/or ceruminosis associated diseases, through direct application of the otic compositions and formulations to the external ear canal.

### BACKGROUND OF THE INVENTION

Cerumen, also known as external auditory canal (“EAC”) wax or earwax, is a yellowish waxy substance secreted in the ear canal. Cerumen aids in cleaning and lubricating the skin and protects the vulnerable skin area from potentially harmful organisms.

### SUMMARY OF THE INVENTION

Disclosed herein are pharmaceutical compositions suitable for local administration to the EAC for modulating the production of cerumen. In some embodiments, the composition includes an otic agent for modulating the production of cerumen; and an auris-acceptable gel.

In some embodiment, the auris-acceptable gel is an aqueous auris-acceptable gel. In some embodiments, the auris-acceptable gel is an auris external-acceptable gel.

In some embodiments, the auris external-acceptable gel is an auris-acceptable thermoreversible gel. In some embodiments, the composition has a gelation temperature between about 19° C. to about 42° C.

In some embodiments, the composition has an apparent viscosity of about 15,000 cP to about 1,000,000 cP. In some embodiments, the composition has an apparent viscosity of about 100,000 cP to about 500,000 cP. In some embodiments, the composition has an apparent viscosity of about 250,000 cP to about 500,000 cP.

In some embodiments, the composition has a practical osmolarity between about 150 to about 500 mOsm/L. In some embodiments, the composition has a practical osmolarity between about 200 to about 400 mOsm/L. In some embodiments, the composition has a practical osmolarity between about 250 to about 320 mOsm/L.

In some embodiments, the otic agent has a mean dissolution time of about 30 hours.

In some embodiments, the otic agent is released from the composition over a period of at least 3 days. In some embodiments, the otic agent is released from the composition over a period of at least 4 days. In some embodiments, the otic agent is released from the composition over a period of at least 5 days. In some embodiments, the otic agent is released from the composition over a period of at least 7 days. In some embodiments, the otic agent is released from the composition over a period of at least 14 days.

In some embodiments, the otic agent is in the form of a neutral molecule, free acid, free base, a salt, a prodrug, or a combination thereof.

In some embodiments, the otic agent comprises multiparticulates. In some embodiments, the otic agent is essentially in the form of micronized particles. In some embodiments, the otic agent is in the form of micronized particles.

In some embodiments, the pH of the composition is between about 5.5 to about 9.0 In some embodiments, the pH of the composition is between about 6.0 to about 8.5. In some embodiments, the pH of the composition is between about 7.0 to about 8.0.

In some embodiments, the composition is essentially free of alcohol solvent. In some embodiments, the composition is essentially free of glycol solvent.

In some embodiments, the auris-acceptable gel is bioerodable.

In some embodiments, the otic agent is choline ester or carbamate, plant alkaloid, reversible cholinesterase inhibitor, acetylcholine release promoter, anti-adrenergy, sympathomimetic, or a combination thereof. In some embodiments, the otic agent is choline ester or carbamate, preferrably acetylcholine or carbachol. In some embodiments, the otic agent is plant alkaloid, preferably pilocarpine. In some embodiments, the otic agent is reversible cholinesterase inhibitor, preferably neostigmine or physostigmine. In some embodiments, the otic agent is acetylcholine release promoter, preferably droperidol, resperidone, or trazodone. In some embodiments, the otic agent is anti-adrenergic, preferably clonidine, propranolol, atenolol, or prazosin. In some embodiments, the otic agent is sympathomimetic, preferably norepinephrine, or dopamine.

In some embodiments, the composition comprises about 0.1% to about 20% by weight of the otic agent. In some embodiments, the composition comprises about 1% to about 10% by weight of the otic agent. In some embodiments, the composition comprises about 5% to about 8% by weight of the otic agent.

In some embodiments, the composition further comprises one or more EAC protectant. In some embodiments, the EAC protectant is selected from squalene, lanosterol, and cholesterol. In some embodiments, the EAC protectant is one or more antimicrobial agent. In some embodiments, the antimicrobial agent is an antimicrobial peptide.

In some embodiments, the composition is used in the treatment of ceruminosis. In some embodiments, ceruminosis is associated with a disease or condition. In some embodiments, the disease or condition is ear pruritus, otitis externa, otalgia, tinnitus, vertigo, ear fullness, hearing loss, or a combination thereof.

Also disclosed herein are methods of modulating cerumen production or methods of treating cerumenosis. In some embodiments, the method includes administering to an individual in need thereof a pharmaceutical composition comprising an amount of an otic agent that modulates cerumen production; and an auris-acceptable gel.

In some embodiments, cerumenosis ceruminosis is associated with a disease or condition. In some embodiments, the disease or condition is ear pruritus, otitis externa, otalgia, tinnitus, vertigo, ear fullness, hearing loss, or a combination thereof.

In some embodiments, the composition is administered locally to the external auditory canal, the outer surface of the tympanic membrane, or a combination thereof. In some embodiments, the composition is not administered through the tympanic membrane.

In some embodiments, the methods further include administering an EAC protectant to the individual in need thereof. In some embodiments, the EAC protectant is selected from squalene, lanosterol, and cholesterol. In some embodiments, the EAC protectant is one or more antimicrobial agent. In some embodiments, the antimicrobial agent is an antimicrobial peptide.

In some embodiments, the EAC protectant is incorporated into the pharmaceutical composition comprising the otic agent.

In some embodiments, the EAC protectant is formulated into a supplemental composition administered separately from the pharmaceutical composition comprising the otic agent. In some embodiments, the supplemental composition further comprises an auris-acceptable gel. In some embodiments, the supplemental composition is administered locally to the external auditory canal, the outer surface of the tympanic membrane, or a combination thereof. In some embodiments, the supplemental composition is not administered through the tympanic membrane.

In some embodiments, the pharmaceutical compositions used in the disclosed methods are as summarized above.

In some embodiment of the pharmaceutical composition or method disclosed herein, the pharmaceutical composition does not provide sustained release of otic agent that modulates cerumen production into the middle ear and/or inner ear. In some embodiments of the pharmaceutical composition or method disclosed herein, the pharmaceutical composition does not provide any release of otic agent that modulates cerumen production into the middle ear and/or inner ear.

Other features and technical effects of the methods and compositions described herein will become apparent from the following detailed description. It should be understood, however, that the detailed description and the specific examples, while indicating specific embodiments, are given by way of illustration only.

### BRIEF DESCRIPTION OF FIGURES

FIG. 1 illustrates a comparison of non-sustained release and sustained release formulations.

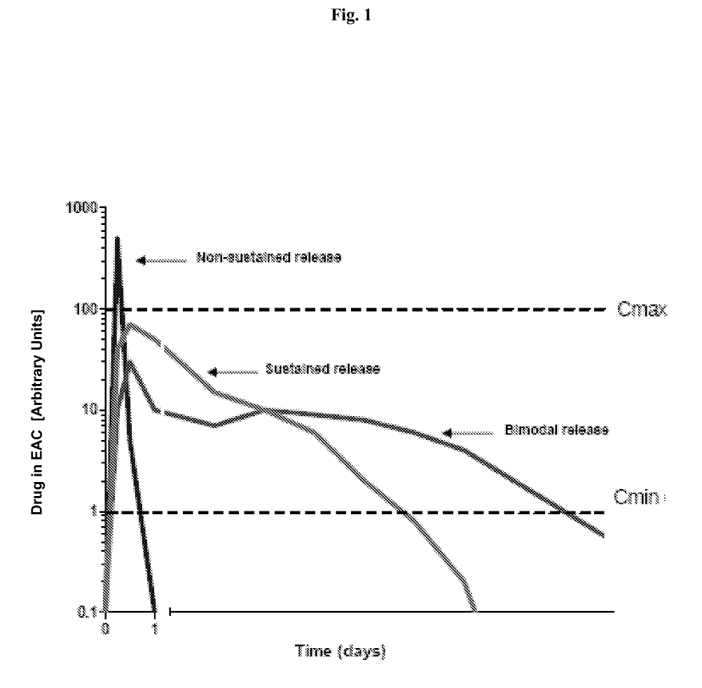

FIG. 2 illustrates the anatomy of the ear.

FIG. 3 shows predicted tunable release of an active agent from four compositions.

### DETAILED DESCRIPTION OF THE INVENTION

Cerumen is an exudate of both the normal and diseased ear canal which, upon accumulation, can disrupt or interfere with normal aural functions, and can even cause discomfort and pain to the patient. Itching, pain, a sense of fullness, noises and even loss of hearing may result from ceruminosis, or cerumen impaction. Occlusion of the ear drum may occur quite suddenly by water entering the ear canal causing the wax to swell. This is frequently the case in individuals who submerge their heads in water during bathing, and tinnitus and even vertigo has been known to result because of the aural pressures developed. Methods of removing cerumen include irrigation, manual removal other than irrigation, cerumenolytic agents for softening cerumen, or a combination thereof. These methods sometimes result in complications such as tympanic membrane perforation, ear canal laceration, infection of the ear, or hearing loss.

The present disclosure recognize the challenges in drug delivery to the EAC. Disclosed herein, in certain embodiments, are compositions, formulations, methods, uses, kits, and delivery devices for modulating the production of cerumen. In certain embodiments, disclosed herein are compositions, formulations, methods, uses, kits, and delivery devices for increasing the production of cerumen. In certain embodiments, disclosed herein are compositions, formulations, methods, uses, kits, and delivery devices for preventing or reducing the build-up or formation of cerumen. In certain embodiments, disclosed herein are compositions, formulations, methods, uses, kits, and delivery devices for removing the build-up or formation of cerumen. In certain embodiments, disclosed herein are compositions, formulations, methods, uses, kits, and delivery devices for treating a cerumen-associated disease or condition, such as ceruminosis. In certain embodiments, disclosed herein are compositions, formulations, methods, uses, kits, and delivery devices for treating ceruminosis-associated diseases or conditions.

Also disclosed herein, are controlled release otic compositions and formulations for modulating the production of cerumen and to treat ceruminosis and ceruminosis associated diseases. The formulations described herein provide a constant, sustained, extended, or delayed rate of release of an active agent into the external ear canal environment and thus avoid any variability in drug exposure in treatment of cerumen production, ceruminosis and ceruminosis associated diseases.

Further provided herein are otic formulations that are sterilized with stringent sterilty requirements and are suitable for administration to the external ear canal. In some embodiments, the auris compatible compositions described herein are substantially free of pyrogens and/or microbes.

Provided herein are otic formulations that meet certain criteria for pH, osmolarity, ionic balance, sterility, endotoxin and/or pyrogen levels. The otic compositions described herein are compatible with the microenvironment of the EAC and are suitable for administration to humans.

By way of non-limiting example, the use of the following commonly used solvents should be limited, reduced or eliminated when formulating agents for administration to the ear: alcohols, propylene glycol, and cyclohexane. Thus, in some embodiments, an otic composition or formulation disclosed herein is free or substantially free of alcohols, propylene glycol, and cyclohexane. In some embodiments, an otic composition or formulation disclosed herein comprises less than about 50 ppm of each of alcohols, propylene glycol, and cyclohexane. In some embodiments, an otic composition or formulation disclosed herein comprises less than about 25 ppm of each of alcohols, propylene glycol, and cyclohexane. In some embodiments, an otic composition or formulation disclosed herein comprises less than about 20 ppm of each of alcohols, propylene glycol, and cyclohexane. In some embodiments, an otic composition or formulation disclosed herein comprises less than about 10 ppm of each of alcohols, propylene glycol, and cyclohexane. In some embodiments, an otic composition or formulation disclosed herein comprises less than about 5 ppm of each of alcohols, propylene glycol, and cyclohexane. In some embodiments, an otic composition or formulation disclosed herein comprises less than about 1 ppm of each of alcohols, propylene glycol, and cyclohexane.

Further, otic preparations require particularly low concentrations of several potentially-common contaminants that are known to be ototoxic. Other dosage forms, while seeking to limit the contamination attributable to these compounds, do not require the stringent precautions that otic preparations require. For example, the following contaminants should be absent or nearly absent from otic preparations: arsenic, lead, mercury, and tin. Thus, in some embodiments, an otic composition or formulation disclosed herein is free or substantially free of arsenic, lead, mercury, and tin. In some embodiments, an otic composition or formulation disclosed herein comprises less than about 50 ppm of each of arsenic, lead, mercury, and tin. In some embodiments, an otic composition or formulation disclosed herein comprises less than about 25 ppm of each of arsenic, lead, mercury, and tin. In some embodiments, an otic composition or formulation disclosed herein comprises less than about 20 ppm of each of arsenic, lead, mercury, and tin. In some embodiments, an otic composition or formulation disclosed herein comprises less than about 10 ppm of each of arsenic, lead, mercury, and tin. In some embodiments, an otic composition or formulation disclosed herein comprises less than about 5 ppm of each of arsenic, lead, mercury, and tin. In some embodiments, an otic composition or formulation disclosed herein comprises less than about 1 ppm of each of arsenic, lead, mercury, and tin.

### Certain Definitions

As used herein, the singular forms “a,” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”, “includes”, and “included”) is not limiting.

As used herein, ranges and amounts can be expressed as “about” a particular value or range. About also includes the exact amount. Hence “about 40 mg” means “about 40 mg” and also “40 mg.” Generally, the term “about” includes an amount that would be expected to be within experimental error.

The term “auris-acceptable” with respect to a formulation, composition or ingredient, as used herein, includes having no persistent detrimental effect on the EAC of the subject being treated. By “auris-pharmaceutically acceptable,” as used herein, refers to a material, such as a carrier or diluent, which does not abrogate the biological activity or properties of the compound in reference to the EAC, and is relatively or is reduced in toxicity to the EAC, i.e., the material is administered to an individual without causing undesirable biological effects or interacting in a deleterious manner with any of the components of the composition in which it is contained.

As used herein, amelioration or lessening of the symptoms of a particular otic disease, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

“Blood plasma concentration” refers to the concentration of compounds provided herein in the plasma component of blood of a subject.

“Carrier materials” are excipients that are compatible with the otic agent, and the release profile properties of the auris-acceptable pharmaceutical formulations. Such carrier materials include, e.g., binders, suspending agents, disintegration agents, filling agents, surfactants, solubilizers, stabilizers, lubricants, wetting agents, diluents, and the like. “Auris-pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

The term “diluent” refers to chemical compounds that are used to dilute the otic agent prior to delivery and which are compatible with the EAC.

“Dispersing agents,” and/or “viscosity modulating agents” are materials that control the diffusion and homogeneity of the otic agent through liquid media. Examples of diffusion facilitators/dispersing agents include but are not limited to hydrophilic polymers, electrolytes, Tween® 60 or 80, PEG, polyvinylpyrrolidone (PVP; commercially known as Plasdone), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., polyoxyethylene-polyoxypropylene triblock copolymers); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol has a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose are also be used as dispersing agents. Dispersing agents useful in liposomal dispersions and self-emulsifying dispersions of the otic agents disclosed herein are dimyristoyl phosphatidyl choline, natural phosphatidyl choline from eggs, natural phosphatidyl glycerol from eggs, cholesterol and isopropyl myristate.

“Drug absorption” or “absorption” refers to the process of movement of the otic agents from the localized site of administration, by way of example only, the EAC. The terms “co-administration” or the like, as used herein, are meant to encompass administration of the otic agents to a single patient, and are intended to include treatment regimens in which the otic agents are administered by the same or different route of administration or at the same or different time.

The terms “effective amount” or “therapeutically effective amount,” as used herein, refer to a sufficient amount of the otic agent being administered that would be expected to relieve to some extent one or more of the symptoms of the disease or condition being treated. For example, the result of administration of the otic agent disclosed herein is reduction and/or alleviation of the signs, symptoms, or causes of ceruminosis. For example, an “effective amount” for therapeutic uses is the amount of an otic agent, including a formulation as disclosed herein required to provide a decrease or amelioration in disease symptoms without undue adverse side effects. The term “therapeutically effective amount” includes, for example, a prophylactically effective amount. An “effective amount” of a modulator of at least one otic agent composition disclosed herein is an amount effective to achieve a desired pharmacologic effect or therapeutic improvement without undue adverse side effects. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician. It is also understood that “an effective amount” in an extend-release dosing format may differ from “an effective amount” in an immediate-release dosing format based upon pharmacokinetic and pharmacodynamic considerations.

The terms “enhance” or “enhancing” refers to an increase or prolongation of either the potency or duration of a desired effect of an otic agent, or a diminution of any adverse symptomatology that is consequent upon the administration of the therapeutic agent. Thus, in regard to enhancing the effect of the otic agents disclosed herein (e.g., sirtuin modulating agents), the term “enhancing” refers to the ability to increase or prolong, either in potency or duration, the effect of other therapeutic agents that are used in combination with the otic agent disclosed herein. An “enhancing-effective amount,” as used herein, refers to an amount of an otic agent or other therapeutic agent which is adequate to enhance the effect of another therapeutic agent or otic agent of the target auris structure in a desired system. When used in a patient, amounts effective for this use will depend on the severity and course of the disease, disorder or condition, previous therapy, the patient's health status and response to the drugs, and the judgment of the treating physician.

The term “inhibiting” includes preventing, slowing, or reversing the development of a condition, for example, or advancement of a condition in a patient necessitating treatment.

“Balance disorder” refers to a disorder, illness, or condition which causes a subject to feel unsteady, or to have a sensation of movement. Included in this definition are dizziness, vertigo, disequilibrium, and pre-syncope. Diseases which are classified as balance disorders include, but are not limited to, hearing loss, dizziness, vertigo, tinnitus and similar conditions

The terms “kit” and “article of manufacture” are used as synonyms.

“Pharmacodynamics” refers to the factors which determine the biologic response observed relative to the concentration of drug at the desired site of the EAC.

“Pharmacokinetics” refers to the factors which determine the attainment and maintenance of the appropriate concentration of drug at the desired site of the EAC.

In prophylactic applications, compositions containing the otic agent described herein are administered to a patient susceptible to or otherwise at risk of a particular disease, disorder or condition, for example, ceruminosis, or patients that are suffering from a disease or symptoms known to be characteristic of ceruminosis, including by way of example only, hearing loss, dizziness, vertigo, and tinnitus. Such an amount is defined to be a “prophylactically effective amount or dose.” In this use, the precise amounts also depend on the patient's state of health, weight, and the like.

The term “substantially low degradation products” means less than 5% by weight of the active agent are degradation products of the active agent. In further embodiments, the term means less than 3% by weight of the active agent are degradation products of the active agent. In yet further embodiments, the term means less than 2% by weight of the active agent are degradation products of the active agent. In further embodiments, the term means less than 1% by weight of the active agent are degradation products of the active agent. In some embodiments, any individual impurity (e.g., metal impurity, degradation products of active agent and/or excipients, or the like) present in a formulation described herein is less than 5%, less than 2%, or less than 1% by weight of the active agent. In some embodiments the formulation does not contain precipitate during storage or change in color after manufacturing and storage.

As used herein “essentially in the form of micronized powder” includes, by way of example only, greater than 70% by weight of the active agent is in the form of micronized particles of the active agent. In further embodiments, the term means greater than 80% by weight of the active agent is in the form of micronized particles of the active agent. In yet further embodiments, the term means greater than 90% by weight of the active agent is in the form of micronized particles of the active agent. The term “micronized” refers to the size of the particles as described herein, and does not limit the particles by the process of its manufacturing. In other words, the “micronized” particles should cover both particles obtained through size-reduction and particles obtained without size-reduction.

The mean residence time (MRT) is the average time that molecules of an active agent reside in an otic structure after a dose.

A “prodrug” refers to an otic agent that is converted into the parent drug in vivo. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In one embodiment, the prodrug is designed to alter the metabolic stability or the transport characteristics of a drug, to mask side effects or toxicity, or to alter other characteristics or properties of a drug. Compounds provided herein, in some embodiments, are derivatized into suitable prodrugs.

“Solubilizers” refer to auris-acceptable compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like that assist or increase the solubility of the otic agents disclosed herein.

“Stabilizers” refers to compounds such as any antioxidation agents, buffers, acids, preservatives and the like that are compatible with the environment of the EAC. Stabilizers include but are not limited to agents that will do any of (1) improve the compatibility of excipients with a container, or a delivery system, including a syringe or a glass bottle, (2) improve the stability of a component of the composition, or (3) improve formulation stability.

“Steady state,” as used herein, is when the amount of drug administered to the EAC is equal to the amount of drug eliminated within one dosing interval resulting in a plateau or constant levels of drug exposure within the targeted structure.

As used herein, the term “subject” is used to mean an animal, preferably a mammal, including a human or non-human. The terms patient and subject may be used interchangeably.

“Surfactants” refer to compounds that are auris-acceptable, such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants are included to enhance physical stability or for other purposes.

The terms “treat,” “treating” or “treatment,” as used herein, include alleviating, abating or ameliorating a disease or condition, for example ceruminosis, symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Cerumen

Cerumen, or earwax, is a waxy secretion found throughout the external ear canal (EAC). Generally, cerumen is stratified into two phenotypes, wet and dry. The wet phenotype has a honey-brown to dark-brown appearance and is characterized by a high concentration of lipid and pigment granules. In some embodiments, the wet cerumen contains about 50% lipid. It is predominantly found in the African and European population. The dry phenotype has a gray to white flaky appearance and is characterized by a low concentration of lipid and pigment granules. In some embodiments, the dry cerumen contains about 20% lipid. It is predominantly found in the Asian and Native American population. Further, these two types of cerumen are genetically distinct, in which a single genetic change in the ATP-binding cassette C11 (ABCC11) gene on chromosome 16 determines the type. Specifically, the allele for the wet phenotype contains a G at 538 of the coding region of ABCC11 whereas for the dry phenotype, an A at 538 is present.

Cerumen lubricates the sensitive ear canal lining from dryness and protects the ear from bacteria, fungi, insects, and foreign particles. Indeed, in several studies, the antimicrobial property of cerumen was demonstrated when the occurrences of ear infections were consistently correlated to absences of cerumen. In some embodiments, cerumen exerts an antimicrobial property against bacteria and fungi. Exemplary bacteria include, but are not limited to, Haemophilus influenza, Staphylococcus aureus, Pseudomonas aeruginosa, and Escherichia coli. Exemplary fungi include, but are not limited to, Aspergillus niger, and Candida albicans.

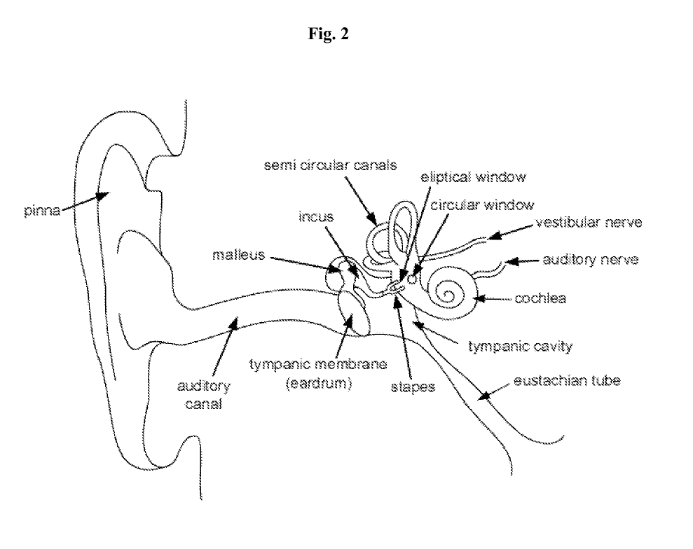

Cerumen is a mixture comprised of over 40 different substances. The primarily component of cerumen is keratin, which comprises about 60% by weight. Additional components include secretions from sebaceous and ceruminous glands, gradular secretions from hairs within the external ear canal (EAC), sloughed epithelial cells, saturated and unsaturated long-chain fatty acids, alcohols, squalene, lanosterol, and cholesterol. The EAC comprise of the pinna (auricle or the fleshy part of the external ear visible on the side of the head), the auditory canal (external auditory meatus) and the outward facing portion of the tympanic membrane, also known as the ear drum (FIG. 2). Cerumen is found throughout the EAC.

Sebaceous glands and ceruminous glands (or modified apocrine glands) are two exocrine glands located in the EAC. Sebaceous glands are exocrine glands located in the skin. They secrete sebum, a viscous oily or waxy secretion, which is used to lubricate and waterproof the skin and hair. There are two types of sebaceous glands, those that connect to hair follicles and those that exist independently. When the sebaceous glands are connected to hair follicles, the deposited sebum is secreted onto the base of the hair and then transported onto the surface of the skin via the hair shaft.

Sebaceous glands are known to participate in innate immunity and participate in pro-and anti-inflammatory functions. Sebum, the product of sebaceous glands, has been shown to exert antimicrobial properties as well. Sebum comprises triglycerides, wax esters, squalene, cholesterol esters, cholesterol, and fatty acids such as sapienic acid. Sebum also contains free fatty acids (FFA) which has been shown to exhibit antibacterial activity against a broad range of Gram-positive bacteria in vitro. Further fatty acids such as monoenoic fatty acids (e.g. oleic and palmitoleic acids) have also been shown to exert antibacterial activities. Indeed, administration of palmitoleate was shown to decrease the size of bacterial lesions in wild-type C57BL/6 and mutant flake mice. In a separate study, oleic and palmitoleic acids were shown to be inhibitory against S. aureus and S. pyogenes. In addition to fatty acids, sebaceous glands also release antimicrobial peptides (AMPs) such as human β-defensins (hBDs) including hBD-1, hBD-2, and hBD-3, and LL-37, a 37-amino acid long C-terminal portion of cathelicidin antimicrobial peptide 18 (hCAP-18), which further contribute to the antimicrobial properties in cerumen.

Ceruminous glands or modified apocrine sweat glands are specialized sudoriferous glands located subcutaneously in the external auditory canal. The ceruminous glands comprise an inner secretory layer of cells that form into coiled tubular shaped glands and an outer myoepithelial layer of cells. The glands drain into larger ducts which then drain into the guard hairs residing in the external auditory canal. Ceruminous gland secretes a comparatively less-viscous secretion than the sebum.

Abnormal cerumen occurs when there is an imbalance in the production and elimination mechanisms. A build-up of cerumen can lead to from discomfort to serious health complications.

Disclosed herein, in certain embodiments, are compositions, formulations, methods, uses, kits, and delivery devices for modulating the production of cerumen. In some embodiments, disclosed herein are compositions, formulations, methods, uses, kits, and delivery devices for increasing the production of cerumen. In some embodiments, disclosed herein are compositions, formulations, methods, uses, kits, and delivery devices for preventing the build-up or formation of cerumen. In some embodiments, disclosed herein are compositions, formulations, methods, uses, kits, and delivery devices for removing the build-up or formation of cerumen. In some embodiments, disclosed herein are compositions, formulations, methods, uses, kits, and delivery devices for treating a cerumen-associated disease or condition, such as ceruminosis.

### Ceruminosis

Ceruminosis or cerumen impaction occurs when earwax becomes wedged in and blocks the EAC and/or impaction on the eardrum. Ceruminosis occurs in about one in 10 children, one in 20 adults, and more than one-third of the geriatric and developmentally delayed populations. About 12 million people seek medical care annually in the United States. In some embodiments, impaction of cerumen is a complete obstruction of the EAC. In some embodiments, impaction of cerumen is a partial obstruction of the EAC.

The occurrences of ceruminosis can be attributed to a build-up of cerumen in the EAC, normal extrusion such as hearing aides leading to compounded cerumen, or by the use of cotton buds or other ear cleaning devices which compounds cerumen. Disease or conditions associated with ceruminosis include ear pruritus, otalgia, tinnitus, vertigo, ear fullness, and hearing loss.

Treatments for ceruminosis include irrigation, manual removal other than irrigation, cerumenolytic agents for softening cerumen, or a combination thereof. Irrigation includes the use of water or saline solution by ear syringing. Manual removal other than irrigation involves the use of curette, probe, hook, forceps, or suction. Cerumenolytic agents include water-based, oil-based, and non-water-, non-oil based agents. For example, water-based cerumenolytic agents include acetic acid, CERUMENEX® (triethanolamine polypeptide oleate condensate), COLACE® (docusate sodium), MOLCER® (docusate sodium), WAXSOL1® (docusate sodium, mixed parabens in 2-phenoxyethanol), XERUMENEX® (triethanolamine polypeptide oleate-condensate, propylene glycol, and chlorbutol), hydrogen peroxide, sodium bicarbonate, and sterile saline solution. Oil-based cerumenolytic agents include almond oil, arachis oil, olive oil, a mineral oil/liquid petrolatum combination, CLEANEARS® (a composition of mineral oil, squalene and spiramint oil), CERUMOL® (a compositon of arachis oil, turpentine oil, chlorbutol, and paradichlorobenzene), CIOCTYL-MEDO® (dioctyl sodium sulphosuccinate, maize oil), and EAREX® (archis oil, almond oil, and rectified camphor oil). Non-water-, non-oil-based cerumenolytic agents include AUDAX® (choline salicylate, glycerine), DEBROX® (carbamide peroxide), AURO® (a composition of carbamide peroxide and anhydrous glycerin) and EXTEROL® (carbamide peroxide and anhydrous glycerol).

Sometimes, treatments of ceruminosis result in significant complications. For example, complications such as tympanic membrane perforation, ear canal laceration, infection of the ear, or hearing loss occur at a rate of about one in 1000 ear irrigations. Additional complications include otitis externa, pain, dizziness and syncope or fainting. The present disclosure recognizes the need for otic compositions and treatment methods that reduces or ameliorates the complications associated with cerumen removal.

Disclosed herein, in certain embodiments, are compositions, formulations, methods, uses, kits, and delivery devices for treating ceruminosis comprising administering to an individual in need thereof a composition comprising an amount of an otic agent and an aqueous auris-acceptable gel. Further disclosed herein, in certain embodiments, are compositions, formulations, methods, uses, kits, and delivery devices for treating ceruminosis associated diseases or conditions comprising administering to an individual in need thereof a composition comprising an amount of an otic agent and an aqueous auris-acceptable gel.

Ceruminosis Associated Diseases or Conditions

Diseases or conditions associated with ceruminosis include ear pruritus, otitis externa, otalgia, tinnitus, vertigo, ear fullness, and hearing loss. Disclosed herein are compositions and methods that modulate the production of cerumen and thereby alleviate the diseases or conditions described herein.

Ear Pruritus

Ear pruritus, or itchy ear canal, is a tickling or irritating sensation that causes a desire or reflex to scratch the affected area. In some cases, redness, swelling, soreness and flaking may develop in the affected area. Ear pruritus is caused by a variety of agents. In some embodiments, ear pruritus occurs due to either primary microbial infection within the ear or as a secondary infection from the body where it is then spread into the ear canal. In some embodiments, skin conditions such as eczema or psoriasis lead to skin irritations within the ear canal. Further, external irritants such as hairspray, shampoo, shower gel, or allergen such as dust, pets, and pollen, can lead to ear pruritus. In some embodiments, ear pruritus serves as an early sign for more serious complications such as otitis externa.

Otitis Externa

Otitis externa is an inflammation of the external auditory canal. It is accompanied by otalgia (ear pain or discomfort) and otorrhea (discharge in or coming from the external auditory canal). Further, if inflammation induces sufficient swelling to occlude the external auditory canal, aural fullness and loss of hearing may also occur. Otitis externa is classified into two types, chronic otitis externa and acute otitis externa (AOE). AOE is predominantly due to bacterial or fungal infection. However, it can also be associated with noninfectious systemic or local dermatologic processes such as atopic dermatitis, psoriasis, seborrheic dermatitis, acne, and lupus erythematosus. In some embodiments, Pseudomonas aeruginosa and Staphylococcus aureus have been know to be the primary bacterial source of infection while Candida albicans and Aspergillus species are the fungal counterparts. In general, topical solutions containing drying agents and/or antibiotics are prescribed for mild cases. However in severe cases, systemic analgesics such as codeine and non-steroidal anti-inflammatory drugs (NSAIDs) might be required.

Otalgia

Otalgia, also known as earache or ear pain, is classified into two types, primary otalgia and referred otalgia. Primary otalgia is ear pain which originates from inside of the ear. Referred otalgia is ear pain which originates from the outside of the ear. Although the etiology of referred otalgia can be complex, several well-known culprits include dental disorders, sinusitis, neck problems, tonsillitis, pharyngitis, and sensory branches from the vagus and glossopharyngeal nerves. In some cases, referred otalgia has been associated with head and neck malignancies.

Ear Fullness

Ear fullness or aural fullness is described as a feeling that the ears are clogged, stuffed, or congested. Similar to otalgia, the etiology of ear fullness is diverse with numerous underlying causes. Generally, ear fullness may also be accompanied by tinnitus, otalgia, and impaired hearing.

Hearing Loss

Hearing loss is a partial or total impairment to hear. Hearing loss can be classified into three types, conductive hearing loss, sensorineural hearing loss, and mixed hearing loss. Conductive hearing loss occurs when sound is not conducted efficiently through the external auditory canal to the tympanic membrane or eardrum. In some embodiments, conductive hearing loss involves a reduction in sound level or the ability to hear faint sounds. Treatment involves corrective medical or surgical procedures. Sensorineural hearing loss occurs when there is damage to the cochlea (inner ear), or to the nerve pathways from the cochlea to the brain. This type of hearing loss generally leads to permanent hearing loss. Mixed hearing loss is a combination of conductive hearing loss and sensorineural hearing loss in which damage occurs along both the outer and inner ear regions.

The degree or severity of hearing loss is categorized into seven groups ranging from normal, slight, mild, moderate, moderately severe, severe to pround. In addition, hearing loss can be stratified based on frequency. For example, a hearing loss that only affects the high tones is referred to as a high frequency hearing loss, whereas that which affects the low tones is referred to as a low frequency hearing loss. In some cases, hearing loss affects both high and low frequencies.

Hearing loss is often accompanied by additional causes and symptoms such as ceruminosis, otitis externa, otalgia, tinnitus and vertigo. In some embodiments, it has been shown that ceruminosis can decrease hearing acuity by 40-45 dB. Such impairment, especially in the geriatic population can cause difficulties in communication and even physical immobility.

Tinnitus

Tinnitus is defined as the perception of sound in the absence of any external stimuli. It may occur in one or both ears, continuously or sporadically, and is most often described as a ringing sound. It is most often used as a diagnostic symptom for other diseases. There are two types of tinnitus: objective and subjective. The former is a sound created in the body which is audible to anyone. The latter is audible only to the affected individual. Studies estimate that over 50 million Americans experience some form of tinnitus. Of those 50 million, about 12 million experience severe tinnitus.

There are several treatments for tinnitus. Lidocaine, administered by IV, reduces or eliminates the noise associated with tinnitus in about 60-80% of sufferers. Selective neurotransmitter reuptake inhibitors, such as nortriptyline, sertraline, and paroxetine, have also demonstrated efficacy against tinnitus. Benzodiazepines are also prescribed to treat tinnitus.

Vertigo

Vertigo is described as a feeling of spinning or swaying while the body is stationary. There are two types of vertigo. Subjective vertigo is the false sensation of movement of the body. Objective vertigo is the perception that one's surrounding are in motion. It is often accompanied by nausea, vomiting, and difficulty maintaining balance. In some embodiments, otitis externa can induce vertigo.

### Pharmaceutical Agents

Provided herein are compositions or formulations that modulate the production of cerumen. Also provided herein are compositions or formulations that modulate the function or activity of the exocrine glands disclosed herein. Further provided herein are compositions or formulations that ameliorate or lessen ceruminosis. In addition, provided herein are compositions or formulations that ameliorate or lessen ceruminosis associated disorders including ear pruritus, otitis externa, otalgia, tinnitus, vertigo, ear fullness, and hearing loss.

Cerumen, ceruminosis and ceruminosis associated disorders exhibit causes and symptoms that are responsive to the pharmaceutical agents disclosed herein. Otic agents which are not disclosed herein but which are useful for the amelioration or eradication of cerumen and ceruminosis associated disorders are expressly included and intended within the scope of the embodiments presented.

In some embodiments, otic agents include choline ester or carbamate, plant alkaloid, reversible cholinesterase inhibitor, acetylcholine release promoter, anti-adrenergy, sympathomimetic, or a combination thereof. In some embodiments, the otic agent is choline ester or carbamate, plant alkaloid, reversible cholinesterase inhibitor, acetylcholine release promoter, anti-adrenergy, sympathomimetic, or a combination thereof. In some embodiments, the otic agent is choline ester or carbamate, preferrably acetylcholine or carbachol. In some embodiments, the otic agent is plant alkaloid, preferably pilocarpine. In some embodiments, the otic agent is reversible cholinesterase inhibitor, preferably neostigmine or physostigmine. In some embodiments, the otic agent is acetylcholine release promoter, preferably droperidol, resperidone, or trazodone. In some embodiments, the otic agent is anti-adrenergic, preferably clonidine, propranolol, atenolol, or prazosin. In some embodiments, the otic agent is sympathomimetic, preferably norepinephrine, or dopamine.

In some embodiments, pharmaceutical agents which have been previously shown to be toxic, harmful or non-effective during systemic or localized application in other organ systems, for example through toxic metabolites formed after hepatic processing, toxicity of the drug in particular organs, tissues or systems, through high levels needed to achieve efficacy, through the inability to be released through systemic pathways or through poor pK characteristics, are useful in some embodiments herein. Accordingly, pharmaceutical agents which have limited or no systemic release, systemic toxicity, poor pK characteristics or combinations thereof are contemplated within the scope of the embodiments disclosed herein.

Formulations comprising otic agents disclosed herein are optionally targeted directly to otic structures where treatment is needed. In some embodiments, application of the otic agent comprising formulations disclosed herein is applied to the external auditory canal, the outer surface of the tympanic membrane, or a combination thereof. Such embodiments also optionally comprise a drug delivery device, wherein the drug delivery device delivers the disclosed formulations through use of a syringe and/or needle, a pump, dropper, an in situ forming hydrogel material, or any combination thereof.

Optionally, a controlled release otic formulation includes otoprotective agents, such as antioxidants, alpha lipoic acid, calcium, fosfomycin or iron chelators, to counteract potential ototoxic effects that may arise from the use of specific therapeutic agents or excipients, diluents or carriers.

### EAC Protectant

Exocrine Gland Secreted Agents

Exocrine gland secretions and exocrine gland secreted agents are contemplated for use with the formulations disclosed herein. Accordingly, some embodiments incorporate the use of secreted agents that mimic the natural cerumen composition and/or exert antimicrobial properties.

Exocrine gland is classified into three categories, holocrine glands, merocrine (or eccrine) glands, and apocrine glands. Holocrine glands accumulate their secretions into each cell's cytoplasm and release the whole cell into the duct. Sebaceous gland is an example of a holocrine gland. Apocrine glands are sweat glands, with ceruminous gland as an example.

Sebum is the product secreted from the sebaceous gland. In some embodiments, sebum comprises triglycerides, wax esters, squalene, cholesterol esters, cholesterol, and fatty acids. In some embodiments, sebum comprises squalene, lanosterol and cholesterol. Squalene which is secreted as part of sebum serves as a precursor for all animal steroids including lanosterol and cholesterol. Squalene is produced via the mevalonate pathway which is responsible for the production of cholesterol and other isoprenoids. HMG-CoA (or 3-hydroxy-3-methylglutaryl-coenzyme A) reductase is the rate-controlling enzyme in the mevalonate pathway.

In some embodiments, the exocrine gland secreted agents comprise at least one of triglycerides, wax esters, squalene, cholesterol esters, cholesterol, and fatty acids. In some embodiments, the exocrine gland secreted agents comprise at least one of squalene, lanosterol and cholesterol. In some embodiments, the components of cerumen comprise the exocrine gland secreted agents. In some embodiments, cerumen comprises at least one of triglycerides, wax esters, squalene, cholesterol esters, cholesterol, and fatty acids. In some embodiments, cerumen comprises at least one of squalene, lanosterol and cholesterol. In some embodiments, the otic composition disclosed herein further comprises an additional active agent. In some embodiments, the additional active agent comprises at least one of triglycerides, wax esters, squalene, cholesterol esters, cholesterol, and fatty acids. In some embodiments, the additional active agent comprises at least one of squalene, lanosterol and cholesterol. In some embodiments, the otic composition further comprises at least one of triglycerides, wax esters, squalene, cholesterol esters, cholesterol, and fatty acids. In some embodiments, the otic composition further comprises at least one of squalene, lanosterol and cholesterol. In some embodiments, the otic composition further comprises squalene, lanosterol and cholesterol.

In some embodiments, the percentage by weight of squalene is from about 1% to about 20%. In some embodiments, the percentage by weight of squalene is from about 2% to about 15%. In some embodiments, the percentage by weight of squalene is from about 3% to about 10%. In some embodiments, the percentage by weight of squalene is from about 5% to about 8%. In some embodiments, the percentage by weight of squalene is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In some embodiments, the percentage by weight of lanosterol is from about 1% to about 20%. In some embodiments, the percentage by weight of lanosterol is from about 2% to about 15%. In some embodiments, the percentage by weight of lanosterol is from about 3% to about 10%. In some embodiments, the percentage by weight of lanosterol is from about 5% to about 8%. In some embodiments, the percentage by weight of lanosterol is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In some embodiments, the percentage by weight of cholesterol is from about 1% to about 20%. In some embodiments, the percentage by weight of cholesterol is from about 2% to about 15%. In some embodiments, the percentage by weight of cholesterol is from about 3% to about 10%. In some embodiments, the percentage by weight of cholesterol is from about 5% to about 8%. In some embodiments, the percentage by weight of cholesterol is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

Antimicrobial Agents

In some embodiments, cerumen comprises agents that exert antimicrobial properties. In some embodiments, these agents include lipids, proteins, and antimicrobial peptides (AMPs). In some embodiments, lipids include fatty acids, cholesterol, waxes, sterols, monoglycerides, diglycerides, triglycerides, and phospholipids. In some embodiments, fatty acids include free fatty acids (FFAs) and unsaturated fatty acids such as oleic acids and palmitoleic acids. In some embodiments, AMPs include hBD-1, hBD-2, hBD-3, and LL-37.

In some embodiments, the otic composition disclosed herein further comprises an antimicrobial agent. In some embodiments, the antimicrobial agent comprises at least one of FFAs, oleic acids, palmitoleic acids, and AMPs. In some embodiments, the antimicrobial agent comprises at least one of FFAs, oleic acids, palmitoleic acids, hBD-1, hBD-2, hBD-3, and LL-37. In some embodiments, the otic composition disclosed herein further comprises at least one of FFAs, oleic acids, palmitoleic acids, and AMPs. In some embodiments, the otic composition disclosed herein further comprises at least one of FFAs, oleic acids, palmitoleic acids, hBD-1, hBD-2, hBD-3, and LL-37

In some embodiments, the percentage by weight of the antimicrobial agent is from about 1% to about 20%. In some embodiments, the percentage by weight of the antimicrobial agent is from about 2% to about 15%. In some embodiments, the percentage by weight of the antimicrobial agent is from about 3% to about 10%. In some embodiments, the percentage by weight of the antimicrobial agent is from about 5% to about 8%. In some embodiments, the percentage by weight of the antimicrobial agent is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

### Combination Therapy

Corticosteroids

Contemplated for use in combination with the otic formulations disclosed herein are corticosteroid agents which reduce or ameliorate symptoms or effects as a result of an autoimmune disease and/or inflammatory disorder. Such steroids include prednisolone, dexamethasone, beclomethasone, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone phosphate, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide and combinations thereof.

Anti-Emetic Agents/Central Nervous System Agents

Anti-Emetic agents are optionally used in combination with the otic agent formulations disclosed herein. Anti-emetic agents include antihistamines and central nervous agents, including anti-psychotic agents, barbiturates, benzodiazepines and phenothiazines. Other anti-emetic agents include the serotonin receptor antagonists, which include dolasetron, granisetron, ondansetron, tropisetron, palonosetron, and combinations thereof; dopamine antagonists, including domperidone, properidol, haloperidol, chlorpromazine, promethazine, prochlorperazine and combinations thereof; cannabinoids, including dronabinol, nabilone, sativex, and combinations thereof; anticholinergics, including scopolamine; and steroids, including dexamethasone; trimethobenzamine, emetrol, propofol, muscimol, and combinations thereof.

Optionally, Central Nervous System agents and barbiturates are useful in the treatment of nausea and vomiting symptoms that accompany an otic disorder. When used, an appropriate barbiturate and/or central nervous system agent is selected to relieve or ameliorate specific symptoms without possible side effects, including ototoxicity. Moreover, as discussed above, targeting of the drugs to the EAC reduces possible side effects and toxicity caused by systemic administration of these drugs. Barbiturates, which act as a central nervous system depressant, include allobarbital, alphenal, amobarbital, aprobarbital, barnexaclone, barbital, brallobarbital, butabarbital, butalbital, butallylonal, butobarbital, corvalol, crotylbarbital, cyclobarbital, cyclopal, ethallobarbital, febarbamate, heptabarbital, hexethal, hexobarbital, metharbital, methohexital, methylphenobarbital, narcobarbital, nealbarbital, pentobarbital, phenobarbital, primidone, probarbital, propallylonal, proxibarbital, reposal, secobarbital, sigmodal, sodium thiopental, talbutal, thialbarbital, thiamylal, thiobarbital, thiobutabarbital, tuinal, valofane, vinbarbital, vinylbital, and combinations thereof.

Other central nervous system agents which are optionally used in conjunction with the otic agent formulations disclosed herein include benzodiazepines or phenothiazines. Useful benzodiazepines include, but are not limited to diazepam, lorazepam, oxazepam, prazepam, alprazolam, bromazepam, chlordiazepoxide, clonazepam, clorazepate, brotizolam, estazolam, flunitrazepam, flurazepam, loprazolam, lormetazepam, midazolam, nimetazepam, nitrazepam, ternazepam, triazolam, and combinations thereof. Examples of phenothiazines include prochlorperazine, chlorpromazine, promazine, triflupromazine, levopromazine, methotrimepramazine, mesoridazine, thiroridazine, fluphenazine, perphenazine, flupentixol, trifluoperazine, and combinations thereof.

Antihistamines, or histamine antagonists, act to inhibit the release or action of histamine. Antihistamines that target the H1 receptor are useful in the alleviation or reduction of nausea and vomiting symptoms that are associated with otic disorders. Such antihistamines include, but are not limited to, meclizine, diphenhydramine, loratadine and quetiapine. Other antihistamines include mepyramine, piperoxan, antazoline, carbinoxamine, doxylamine, clemastine, dimenhydrinate, pheniramine, chlorphenamine, chlorpheniramine, dexchlorpheniramine, brompheniramine, triprolidine, cyclizine, chlorcyclizine, hydroxyzine, promethazine, alimemazine, trimeprazine, cyproheptadine, azatadine, ketotifen, oxatomide and combinations thereof.

### Concentration of Active Agent

In some embodiments, the compositions described herein have a concentration of active pharmaceutical ingredient between about 0.01% to about 90%, between about 0.01% to about 80%, between about 0.1% to about 70%, between about 0.1% to about 60%, between about 0.1% to about 50%, between about 0.1% to about 40%, between about 0.1% to about 30%, between about 0.1% to about 20%, between about 0.1% to about 10%, or between about 0.1% to about 5%, of the active ingredient, or pharmaceutically acceptable prodrug or salt thereof, by weight of the composition. In some embodiments, the compositions described herein have a concentration of active pharmaceutical agent between about 1% to about 50%, between about 5% to about 50%, between about 10% to about 40%, or between about 10% to about 30%, of the active ingredient, or pharmaceutically acceptable prodrug or salt thereof, by weight of the composition. In some embodiments, formulations described herein comprise about 70% by weight of an otic agent, or pharmaceutically acceptable prodrug or salt thereof, by weight of the formulation. In some embodiments, formulations described herein comprise about 60% by weight of an otic agent, or pharmaceutically acceptable prodrug or salt thereof, by weight of the formulation. In some embodiments, formulations described herein comprise about 50% by weight of an otic agent, or pharmaceutically acceptable prodrug or salt thereof, by weight of the formulation. In some embodiments, formulations described herein comprise about 40% by weight of an otic agent, or pharmaceutically acceptable prodrug or salt thereof, by weight of the formulation. In some embodiments, formulations described herein comprise about 30% by weight of an otic agent, or pharmaceutically acceptable prodrug or salt thereof, by weight of the formulation. In some embodiments, formulations described herein comprise about 25% by weight of an otic agent, or pharmaceutically acceptable prodrug or salt thereof, by weight of the formulation. In some embodiments, formulations described herein comprise about 20% by weight of an otic agent, or pharmaceutically acceptable prodrug or salt thereof, by weight of the formulation. In some embodiments, formulations described herein comprise about 19% by weight of an otic agent, or pharmaceutically acceptable prodrug or salt thereof, by weight of the formulation. In some embodiments, formulations described herein comprise about 18% by weight of an otic agent, or pharmaceutically acceptable prodrug or salt thereof, by weight of the formulation. In some embodiments, formulations described herein comprise about 17% by weight of an otic agent, or pharmaceutically acceptable prodrug or salt thereof, by weight of the formulation. In some embodiments, formulations described herein comprise about 16% by weight of an otic agent, or pharmaceutically acceptable prodrug or salt thereof, by weight of the formulation. In some embodiments, formulations described herein comprise about 15% by weight of an otic agent, or pharmaceutically acceptable prodrug or salt thereof, by weight of the formulation. In some embodiments, formulations described herein comprise about 14% by weight of an otic agent, or pharmaceutically acceptable prodrug or salt thereof, by weight of the formulation. In some embodiments, formulations described herein comprise about 13% by weight of an otic agent, or pharmaceutically acceptable prodrug or salt thereof, by weight of the formulation. In some embodiments, formulations described herein comprise about 12% by weight of an otic agent, or pharmaceutically acceptable prodrug or salt thereof, by weight of the formulation. In some embodiments, formulations described herein comprise about 11% by weight of an otic agent, or pharmaceutically acceptable prodrug or salt thereof, by weight of the formulation. In some embodiments, formulations described herein comprise about 10% by weight of an otic agent by weight of the formulation, or pharmaceutically acceptable prodrug or salt thereof, by weight of the formulation. In some embodiments, formulations described herein comprise about 9% by weight of an otic agent, or pharmaceutically acceptable prodrug or salt thereof, by weight of the formulation. In some embodiments, formulations described herein comprise about 8% by weight of an otic agent, or pharmaceutically acceptable prodrug or salt thereof, by weight of the formulation. In some embodiments, formulations described herein comprise about 7% by weight of an otic agent, or pharmaceutically acceptable prodrug or salt thereof, by weight of the formulation. In some embodiments, formulations described herein comprise about 6% by weight of an otic agent, or pharmaceutically acceptable prodrug or salt thereof, by weight of the formulation. In some embodiments, formulations described herein comprise about 5% by weight of an otic agent, or pharmaceutically acceptable prodrug or salt thereof, by weight of the formulation. In some embodiments, formulations described herein comprise about 4% by weight of an otic agent, or pharmaceutically acceptable prodrug or salt thereof, by weight of the formulation. In some embodiments, formulations described herein comprise about 3% by weight of an otic agent, or pharmaceutically acceptable prodrug or salt thereof, by weight of the formulation. In some embodiments, formulations described herein comprise about 2.5% by weight of an otic agent, or pharmaceutically acceptable prodrug or salt thereof, by weight of the formulation. In some embodiments, formulations described herein comprise about 2% by weight of an otic agent, or pharmaceutically acceptable prodrug or salt thereof, by weight of the formulation. In some embodiments, formulations described herein comprise about 1.5% by weight of an otic agent, or pharmaceutically acceptable prodrug or salt thereof, by weight of the formulation. In some embodiments, formulations described herein comprise about 1% by weight of an otic agent, or pharmaceutically acceptable prodrug or salt thereof, by weight of the formulation. In some embodiments, formulations described herein comprise about 0.5% by weight of an otic agent, or pharmaceutically acceptable prodrug or salt thereof, by weight of the formulation. In some embodiments, formulations described herein comprise about 0.1% by weight of an otic agent, or pharmaceutically acceptable prodrug or salt thereof, by weight of the formulation. In some embodiments, formulations described herein comprise about 0.01% by weight of an otic agent, or pharmaceutically acceptable prodrug or salt thereof, by weight of the formulation. In some embodiments, the formulations described herein have a concentration of active pharmaceutical ingredient, or pharmaceutically acceptable prodrug or salt thereof, between about 0.1 to about 70 mg/mL, between about 0.5 mg/mL to about 70 mg/mL, between about 0.5 mg/mL to about 50 mg/mL, between about 0.5 mg/mL to about 20 mg/mL, between about 1 mg to about 70 mg/mL, between about 1 mg to about 50 mg/mL, between about 1 mg/mL and about 20 mg/mL, between about 1 mg/mL to about 10 mg/mL, or between about 1 mg/mL to about 5 mg/mL, of the active agent, or pharmaceutically acceptable prodrug or salt thereof, by volume of the formulation.

### General Methods of Sterilization

Provided herein are otic compositions that modulate the production of cerumen, and/or modulate the function or activity of the exocrine glands disclosed herein. Also provided herein are otic compositions that ameliorate or lessen ceruminosis and ceruminosis associated disorders. Further provided herein are methods comprising the administration of the otic compositions disclosed herein. In some embodiments, the compositions are sterilized. Included within the embodiments disclosed herein are means and processes for sterilization of a pharmaceutical composition disclosed herein for use in humans. The goal is to provide a safe pharmaceutical product, relatively free of infection causing micro-organisms. The U. S. Food and Drug Administration has provided regulatory guidance in the publication “Guidance for Industry: Sterile Drug Products Produced by Aseptic Processing” available at: http://www.fda.gov/cder/guidance/5882fnl.htm, which is incorporated herein by reference in its entirety.

As used herein, sterilization means a process used to destroy or remove microorganisms that are present in a product or packaging. Any suitable method available for sterilization of objects and compositions is used. Available methods for the inactivation of microorganisms include, but are not limited to, the application of extreme heat, lethal chemicals, or gamma radiation. In some embodiments is a process for the preparation of an otic therapeutic formulation comprising subjecting the formulation to a sterilization method selected from heat sterilization, chemical sterilization, radiation sterilization or filtration sterilization. The method used depends largely upon the nature of the device or composition to be sterilized. Detailed descriptions of many methods of sterilization are given in Chapter 40 of Remington: The Science and Practice of Pharmacy published by Lippincott, Williams & Wilkins, and is incorporated by reference with respect to this subject matter.

Sterilization by Heat

Many methods are available for sterilization by the application of extreme heat. One method is through the use of a saturated steam autoclave. In this method, saturated steam at a temperature of at least 121° C. is allowed to contact the object to be sterilized. The transfer of heat is either directly to the microorganism, in the case of an object to be sterilized, or indirectly to the microorganism by heating the bulk of an aqueous solution to be sterilized. This method is widely practiced as it allows flexibility, safety and economy in the sterilization process.

Dry heat sterilization is a method which is used to kill microorganisms and perform depyrogenation at elevated temperatures. This process takes place in an apparatus suitable for heating HEPA-filtered microorganism-free air to temperatures of at least 130-180° C. for the sterilization process and to temperatures of at least 230-250° C. for the depyrogenation process. Water to reconstitute concentrated or powdered formulations is also sterilized by autoclave. In some embodiments, the formulations described herein comprise micronized otic agents (e.g., micronized linopirdine powder) that are sterilized by dry heating, e.g., heating for about 7-11 hours at internal powder temperatures of 130-140° C., or for 1-2 hours at internal temperatures of 150-180° C.

Chemical Sterilization

Chemical sterilization methods are an alternative for products that do not withstand the extremes of heat sterilization. In this method, a variety of gases and vapors with germicidal properties, such as ethylene oxide, chlorine dioxide, formaldehyde or ozone are used as the anti-apoptotic agents. The germicidal activity of ethylene oxide, for example, arises from its ability to serve as a reactive alkylating agent. Thus, the sterilization process requires the ethylene oxide vapors to make direct contact with the product to be sterilized.

Radiation Sterilization

One advantage of radiation sterilization is the ability to sterilize many types of products without heat degradation or other damage. The radiation commonly employed is beta radiation or alternatively, gamma radiation from a ⁶⁰Co source. The penetrating ability of gamma radiation allows its use in the sterilization of many product types, including solutions, compositions and heterogeneous mixtures. The germicidal effects of irradiation arise from the interaction of gamma radiation with biological macromolecules. This interaction generates charged species and free-radicals. Subsequent chemical reactions, such as rearrangements and cross-linking processes, result in the loss of normal function for these biological macromolecules. The formulations described herein are also optionally sterilized using beta irradiation.

Filtration

Filtration sterilization is a method used to remove but not destroy microorganisms from solutions. Membrane filters are used to filter heat-sensitive solutions. Such filters are thin, strong, homogenous polymers of mixed cellulosic esters (MCE), polyvinylidene fluoride (PVF; also known as PVDF), or polytetrafluoroethylene (PTFE) and have pore sizes ranging from 0.1 to 0.22 μm. Solutions of various characteristics are optionally filtered using different filter membranes. For example, PVF and PTFE membranes are well suited to filtering organic solvents while aqueous solutions are filtered through PVF or MCE membranes. Filter apparatus are available for use on many scales ranging from the single point-of-use disposable filter attached to a syringe up to commercial scale filters for use in manufacturing plants. The membrane filters are sterilized by autoclave or chemical sterilization. Validation of membrane filtration systems is performed following standardized protocols (Microbiological Evaluation of Filters for Sterilizing Liquids, Vol 4, No. 3. Washington, D.C.: Health Industry Manufacturers Association, 1981) and involve challenging the membrane filter with a known quantity (ca. 10⁷/cm²) of unusually small microorganisms, such as Brevundimonas diminuta (ATCC 19146).

Pharmaceutical compositions are optionally sterilized by passing through membrane filters. Formulations comprising nanoparticles (U.S. Pat. No. 6,139,870) or multilamellar vesicles (Richard et al., International Journal of Pharmaceutics (2006), 312(1-2):144-50) are amenable to sterilization by filtration through 0.22 μm filters without destroying their organized structure.

In some embodiments, the methods disclosed herein comprise sterilizing the formulation (or components thereof) by means of filtration sterilization. In another embodiment the auris-acceptable otic formulation comprises a particle wherein the particle formulation is suitable for filtration sterilization. In a further embodiment said particle formulation comprises particles of less than 300 nm in size, of less than 200 nm in size, of less than 100 nm in size. In another embodiment the auris-acceptable formulation comprises a particle formulation wherein the sterility of the particle is ensured by sterile filtration of the precursor component solutions. In another embodiment the auris-acceptable formulation comprises a particle formulation wherein the sterility of the particle formulation is ensured by low temperature sterile filtration. In a further embodiment, low temperature sterile filtration is carried out at a temperature between 0 and 30° C., between 0 and 20° C., between 0 and 10° C., between 10 and 20° C., or between 20 and 30° C.

In another embodiment is a process for the preparation of an auris-acceptable particle formulation comprising: filtering the aqueous solution containing the particle formulation at low temperature through a sterilization filter; lyophilizing the sterile solution; and reconstituting the particle formulation with sterile water prior to administration. In some embodiments, a formulation described herein is manufactured as a suspension in a single vial formulation containing the micronized active pharmaceutical ingredient. A single vial formulation is prepared by aseptically mixing a sterile poloxamer solution with sterile micronized active ingredient (e.g., choline ester or carbamate, plant alkaloids, reversible cholinesterase inhibitor, acetylcholine release promoter, anti-adrenergic, sympathomimetic) and transferring the formulation to sterile pharmaceutical containers. In some embodiments, a single vial containing a formulation described herein as a suspension is resuspended before dispensing and/or administration.

In specific embodiments, filtration and/or filling procedures are carried out at about 5° C. below the gel temperature (Tgel) of a formulation described herein and with viscosity below a theoretical value of 100 cP to allow for filtration in a reasonable time using a peristaltic pump.

In another embodiment the auris-acceptable otic formulation comprises a nanoparticle formulation wherein the nanoparticle formulation is suitable for filtration sterilization. In a further embodiment the nanoparticle formulation comprises nanoparticles of less than 300 nm in size, of less than 200 nm in size, or of less than 100 nm in size. In another embodiment the auris-acceptable formulation comprises a thermoreversible gel formulation wherein the sterility of the gel formulation is ensured by low temperature sterile filtration. In a further embodiment, the low temperature sterile filtration occurs at a temperature between 0 and 30° C., or between 0 and 20° C., or between 0 and 10° C., or between 10 and 20° C., or between 20 and 30° C. In another embodiment is a process for the preparation of an auris-acceptable thermoreversible gel formulation comprising: filtering the aqueous solution containing the thermoreversible gel components at low temperature through a sterilization filter; lyophilizing the sterile solution; and reconstituting the thermoreversible gel formulation with sterile water prior to administration.

In certain embodiments, the active ingredients are dissolved in a suitable vehicle (e.g. a buffer) and sterilized separately (e.g. by heat treatment, filtration, gamma radiation). In some instances, the active ingredients are sterilized separately in a dry state. In some instances, the active ingredients are sterilized as a suspension or as a colloidal suspension. The remaining excipients (e.g., fluid gel components present in otic formulations) are sterilized in a separate step by a suitable method (e.g. filtration and/or irradiation of a cooled mixture of excipients); the two solutions that are separately sterilized are then mixed aseptically to provide a final otic formulation. In some instances, the final aseptic mixing is performed just prior to administration of a formulation described herein.

In some instances, conventionally used methods of sterilization (e.g., heat treatment (e.g., in an autoclave), gamma irradiation, filtration) lead to irreversible degradation of polymeric components (e.g., thermosetting, or gelling) and/or the active agent in the formulation. In some instances, sterilization of an otic formulation by filtration through membranes (e.g., 0.2 μM membranes) is not possible if the formulation comprises thixotropic polymers that gel during the process of filtration.

Accordingly, provided herein are methods for sterilization of otic formulations that prevent degradation of polymeric components (e.g., thermosetting and/or gelling components) and/or the active agent during the process of sterilization. In some embodiments, degradation of the active agent (e.g., any therapeutic otic agent described herein) is reduced or eliminated through the use of specific pH ranges for buffer components and specific proportions of gelling agents in the formulations. In some embodiments, the choice of an appropriate gelling agent and/or thermosetting polymer allows for sterilization of formulations described herein by filtration. In some embodiments, the use of an appropriate thermosetting polymer and an appropriate copolymer (e.g., a gelling agent) in combination with a specific pH range for the formulation allows for high temperature sterilization of formulations described with substantially no degradation of the therapeutic agent or the polymeric excipients. An advantage of the methods of sterilization provided herein is that, in certain instances, the formulations are subjected to terminal sterilization via autoclaving without any loss of the active agent and/or excipients and/or polymeric components during the sterilization step and are rendered substantially free of microbes and/or pyrogens.

Microorganisms

Provided herein are auris-acceptable compositions that modulate the production of cerumen, and/or modulate the function or activity of the exocrine glands disclosed herein. Also provided herein are otic compositions that ameliorate or lessen ceruminosis and ceruminosis associated disorders. Further provided herein are methods comprising the administration of the otic compositions disclosed herein. In some embodiments, the compositions are substantially free of microorganisms. Acceptable bioburden or sterility levels are based on applicable standards that define therapeutically acceptable compositions, including but not limited to United States Pharmacopeia Chapters <1111> et seq. For example, acceptable sterility (e.g., bioburden) levels include about 10 colony forming units (cfu) per gram of formulation, about 50 cfu per gram of formulation, about 100 cfu per gram of formulation, about 500 cfu per gram of formulation or about 1000 cfu per gram of formulation. In some embodiments, acceptable bioburden levels or sterility for formulations include less than 10 cfu/mL, less that 50 cfu/mL, less than 500 cfu/mL or less than 1000 cfu/mL microbial agents. In addition, acceptable bioburden levels or sterility include the exclusion of specified objectionable microbiological agents. By way of example, specified objectionable microbiological agents include but are not limited to Escherichia coli (E. coli), Salmonella sp., Pseudomonas aeruginosa (P. aeruginosa) and/or other specific microbial agents.

Sterility of the auris-acceptable otic formulation is confirmed through a sterility assurance program in accordance with United States Pharmacopeia Chapters <61>, <62> and <71>. A key component of the sterility assurance quality control, quality assurance and validation process is the method of sterility testing. Sterility testing, by way of example only, is performed by two methods. The first is direct inoculation wherein a sample of the composition to be tested is added to growth medium and incubated for a period of time up to 21 days. Turbidity of the growth medium indicates contamination. Drawbacks to this method include the small sampling size of bulk materials which reduces sensitivity, and detection of microorganism growth based on a visual observation. An alternative method is membrane filtration sterility testing. In this method, a volume of product is passed through a small membrane filter paper. The filter paper is then placed into media to promote the growth of microorganisms. This method has the advantage of greater sensitivity as the entire bulk product is sampled. The commercially available Millipore Steritest sterility testing system is optionally used for determinations by membrane filtration sterility testing. For the filtration testing of creams or ointments Steritest filter system No. TLHVSL210 are used. For the filtration testing of emulsions or viscous products Steritest filter system No. TLAREM210 or TDAREM210 are used. For the filtration testing of pre-filled syringes Steritest filter system No. TTHASY210 are used. For the filtration testing of material dispensed as an aerosol or foam Steritest filter system No. TTHVA210 are used. For the filtration testing of soluble powders in ampoules or vials Steritest filter system No. TTHADA210 or TTHADV210 are used.

Testing for E. coli and Salmonella includes the use of lactose broths incubated at 30-35° C. for 24-72 hours, incubation in MacConkey and/or EMB agars for 18-24 hours, and/or the use of Rappaport medium. Testing for the detection of P. aeruginosa includes the use of NAC agar. United States Pharmacopeia Chapter <62> further enumerates testing procedures for specified objectionable microorganisms.

In certain embodiments, any controlled release formulation described herein has less than about 60 colony forming units (CFU), less than about 50 colony forming units, less than about 40 colony forming units, or less than about 30 colony forming units of microbial agents per gram of formulation. In certain embodiments, the otic formulations described herein are formulated to be isotonic with the EAC.

Endotoxins

Provided herein are otic compositions that modulate the production of cerumen, and/or modulate the function or activity of the exocrine glands disclosed herein. Also provided herein are otic compositions that ameliorate or lessen ceruminosis and ceruminosis associated disorders. Further provided herein are methods comprising the administration of the otic compositions disclosed herein. In some embodiments, the compositions are substantially free of endotoxins. An additional aspect of the sterilization process is the removal of by-products from the killing of microorganisms (hereinafter, “Product”). The process of depyrogenation removes pyrogens from the sample. Pyrogens are endotoxins or exotoxins which induce an immune response. An example of an endotoxin is the lipopolysaccharide (LPS) molecule found in the cell wall of gram-negative bacteria. While sterilization procedures such as autoclaving or treatment with ethylene oxide kill the bacteria, the LPS residue induces a proinflammatory immune response, such as septic shock. Because the molecular size of endotoxins can vary widely, the presence of endotoxins is expressed in “endotoxin units” (EU). One EU is equivalent to 100 picograms of E. coli LPS. Humans can develop a response to as little as 5 EU/kg of body weight. The bioburden (e.g., microbial limit) and/or sterility (e.g., endotoxin level) is expressed in any units as recognized in the art. In certain embodiments, otic compositions described herein contain lower endotoxin levels (e.g. <4 EU/kg of body weight of a subject) when compared to conventionally acceptable endotoxin levels (e.g., 5 EU/kg of body weight of a subject). In some embodiments, the auris-acceptable otic formulation has less than about 5 EU/kg of body weight of a subject. In other embodiments, the auris-acceptable otic formulation has less than about 4 EU/kg of body weight of a subject. In additional embodiments, the auris-acceptable otic formulation has less than about 3 EU/kg of body weight of a subject. In additional embodiments, the auris-acceptable otic formulation has less than about 2 EU/kg of body weight of a subject.

In some embodiments, the auris-acceptable otic formulation has less than about 5 EU/kg of formulation. In other embodiments, the auris-acceptable otic formulation has less than about 4 EU/kg of formulation. In additional embodiments, the auris-acceptable otic formulation has less than about 3 EU/kg of formulation. In some embodiments, the auris-acceptable otic formulation has less than about 5 EU/kg Product. In other embodiments, the auris-acceptable otic formulation has less than about 1 EU/kg Product. In additional embodiments, the auris-acceptable otic formulation has less than about 0.2 EU/kg Product. In some embodiments, the auris-acceptable otic formulation has less than about 5 EU/g of unit or Product. In other embodiments, the auris-acceptable otic formulation has less than about 4 EU/g of unit or Product. In additional embodiments, the auris-acceptable otic formulation has less than about 3 EU/g of unit or Product. In some embodiments, the auris-acceptable otic formulation has less than about 5 EU/mg of unit or Product. In other embodiments, the auris-acceptable otic formulation has less than about 4 EU/mg of unit or Product. In additional embodiments, the auris-acceptable otic formulation has less than about 3 EU/mg of unit or Product. In certain embodiments, otic compositions described herein contain from about 1 to about 5 EU/mL of formulation. In certain embodiments, otic compositions described herein contain from about 2 to about 5 EU/mL of formulation, from about 3 to about 5 EU/mL of formulation, or from about 4 to about 5 EU/mL of formulation.

In certain embodiments, otic compositions described herein contain lower endotoxin levels (e.g. <0.5 EU/mL of formulation) when compared to conventionally acceptable endotoxin levels (e.g., 0.5 EU/mL of formulation). In some embodiments, the auris-acceptable otic formulation or device has less than about 0.5 EU/mL of formulation. In other embodiments, the auris-acceptable otic formulation has less than about 0.4 EU/mL of formulation. In additional embodiments, the auris-acceptable otic formulation has less than about 0.2 EU/mL of formulation.

Pyrogen detection, by way of example only, is performed by several methods. Suitable tests for sterility include tests described in United States Pharmacopoeia (USP)<71> Sterility Tests (23rd edition, 1995). The rabbit pyrogen test and the Limulus amebocyte lysate test are both specified in the United States Pharmacopeia Chapters <85> and <151> (USP23/NF 18, Biological Tests, The United States Pharmacopeial Convention, Rockville, Md., 1995). Alternative pyrogen assays have been developed based upon the monocyte activation-cytokine assay. Uniform cell lines suitable for quality control applications have been developed and have demonstrated the ability to detect pyrogenicity in samples that have passed the rabbit pyrogen test and the Limulus amebocyte lysate test (Taktak et al, J. Pharm. Pharmacol. (1990), 43:578-82). In an additional embodiment, the auris-acceptable otic therapeutic agent formulation is subject to depyrogenation. In a further embodiment, the process for the manufacture of the auris-acceptable otic therapeutic agent formulation comprises testing the formulation for pyrogenicity. In certain embodiments, the formulations described herein are substantially free of pyrogens.

### pH and Practical Osmolarity

In some embodiments, an otic composition disclosed herein is formulated to provide an ionic balance that is compatible with external ear canal (EAC).

In some embodiments, a composition disclosed herein is formulated in order to not disrupt the ionic balance of the external ear canal (EAC). In some embodiments, a composition disclosed herein has an ionic balance that is the same as or substantially the same as the EAC. In some embodiments, a composition disclosed herein does not does not disrupt the ionic balance of the EAC so as to result in complications such as ceruminosis associated conditions.

As used herein, “practical osmolarity/osmolality” or “deliverable osmolarity/osmolality” means the osmolarity/osmolality of a composition as determined by measuring the osmolarity/osmolality of the active agent and all excipients except the gelling and/or the thickening agent (e.g., polyoxyethylene-polyooxypropylene copolymers, carboxymethylcellulose or the like). The practical osmolarity of a composition disclosed herein is measured by a suitable method, e.g., a freezing point depression method as described in Viegas et. al., Int. J. Pharm., 1998, 160, 157-162. In some instances, the practical osmolarity of a composition disclosed herein is measured by vapor pressure osmometry (e.g., vapor pressure depression method) that allows for determination of the osmolarity of a composition at higher temperatures. In some instances, vapor pressure depression method allows for determination of the osmolarity of a composition comprising a gelling agent (e.g., a thermoreversible polymer) at a higher temperature wherein the gelling agent is in the form of a gel.

In some embodiments, the osmolarity at a target site of action (e.g., the EAC) is about the same as the delivered osmolarity of a composition described herein. In some embodiments, a composition described herein has a deliverable osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L.

The practical osmolality of an otic composition disclosed herein is from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a composition described herein has a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L.

In some embodiments, the pH of a composition described herein is adjusted (e.g., by use of a buffer) to an EAC-compatible pH range of about 5.5 to 9.0. In some embodiments, the pH of a composition described herein is adjusted to an EAC-compatible range of about 5.5 to about 8.5, about 6 to about 8.5, about 6.5 to about 8.0, about 6.5 to about 8.0, or about 7.0 to about 8.0. In some embodiments, the pH of a composition described herein is adjusted to an EAC-suitable pH range of about 7.0-7.6.

In some embodiments, useful formulations also include one or more pH adjusting agents or buffering agents. Suitable pH adjusting agents or buffers include, but are not limited to acetate, bicarbonate, ammonium chloride, citrate, phosphate, pharmaceutically acceptable salts thereof and combinations or mixtures thereof.

In one embodiment, when one or more buffers are utilized in the formulations of the present disclosure, they are combined, e.g., with a pharmaceutically acceptable vehicle and are present in the final formulation, e.g., in an amount ranging from about 0.1% to about 20%, from about 0.5% to about 10%. In certain embodiments of the present disclosure, the amount of buffer included in the gel formulations are an amount such that the pH of the gel formulation does not interfere with the body's natural buffering system.

In one embodiment, diluents are also used to stabilize compounds because they can provide a more stable environment. Salts dissolved in buffered solutions (which also can provide pH control or maintenance) are utilized as diluents in the art, including, but not limited to a phosphate buffered saline solution.

In some embodiments, the gel formulation described herein has a pH that allows for sterilization (e.g, by filtration or aseptic mixing or heat treatment and/or autoclaving (e.g., terminal sterilization) of a gel formulation without degradation of the pharmaceutical agent (e.g., otic agent) or the polymers comprising the gel. In order to reduce hydrolysis and/or degradation of the otic agent and/or the gel polymer during sterilization, the buffer pH is designed to maintain pH of the formulation in the 7-8 range during the process of sterilization (e.g., high temperature autoclaving).

In specific embodiments, the gel formulation described herein has a pH that allows for terminal sterilization (e.g, by heat treatment and/or autoclaving) of a gel formulation without degradation of the pharmaceutical agent (e.g., otic agent) or the polymers comprising the gel. For example, in order to reduce hydrolysis and/or degradation of the otic agent and/or the gel polymer during autoclaving, the buffer pH is designed to maintain pH of the formulation in the 7-8 range at elevated temperatures. Any appropriate buffer is used depending on the otic agent used in the formulation. In some instances, since pKₐ of TRIS decreases as temperature increases at approximately −0.03/° C. and pKₐ of PBS increases as temperature increases at approximately 0.003/° C., autoclaving at 250° F. (121° C.) results in a significant downward pH shift (i.e. more acidic) in the TRIS buffer whereas a relatively much less upward pH shift in the PBS buffer and therefore much increased hydrolysis and/or degradation of an otic agent in TRIS than in PBS. Degradation of an otic agent is reduced by the use of an appropriate combination of a buffer and polymeric additives (e.g. CMC) as described herein.

In some embodiments, a formulation pH of between about 5.0 and about 9.0, between about 5.5 and about 8.5, between about 6 and about 8.5, between about 6.5 and about 8.0, between about 6.5 and about 8.0, between about 7.0 to about 8.0, between about 7.0 and about 7.8, between about 7.0 and about 7.6, between about 7.2 and 7.6, or between about 7.2 and about 7.4 is suitable for sterilization (e.g, by filtration or aseptic mixing or heat treatment and/or autoclaving (e.g., terminal sterilization)) of otic formulations described herein. In specific embodiments a formulation pH of about 6.0, about 6.5, about 7.0, about 7.1, about 7.2, about 7.3, about 7.4, about 7.5, or about 7.6 is suitable for sterilization (e.g, by filtration or aseptic mixing or heat treatment and/or autoclaving (e.g., terminal sterilization)) of any composition described herein.

In some embodiments, the formulations have a pH as described herein, and include a thickening agent (e.g, a viscosity enhancing agent) such as, by way of non-limiting example, a cellulose based thickening agent described herein. In some instances, the addition of a secondary polymer (e.g., a thickening agent) and a pH of formulation as described herein, allows for sterilization of a formulation described herein without any substantial degradation of the otic agent and/or the polymer components in the otic formulation. In some embodiments, the ratio of a thermoreversible poloxamer to a thickening agent in a formulation that has a pH as described herein, is about 40:1, about 35:1, about 30:1, about 25:1, about 20:1, about 15:1 about 10:1, or about 5:1. For example, in certain embodiments, a sustained and/or extended release formulation described herein comprises a combination of poloxamer 407 (pluronic F127) and carboxymethylcellulose (CMC) in a ratio of about 40:1, about 35:1, about 30:1, about 25:1, about 20:1, about 15:1, about 10:1 or about 5:1.

In some embodiments, the pharmaceutical formulations described herein are stable with respect to pH over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 1 month, at least about 2 months, at least about 3 months, at least about 4 months, at least about 5 months, or at least about 6 months. In other embodiments, the formulations described herein are stable with respect to pH over a period of at least about 1 week. Also described herein are formulations that are stable with respect to pH over a period of at least about 1 month.

Tonicity Agents

In some embodiments, tonicity agents are added to the formulations described herein in an amount as to provide a practical osmolality of an otic formulation of about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 350 mOsm/kg or from about 280 mOsm/kg to about 320 mOsm/kg. In some embodiments, the formulations described herein have a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 280 mOsm/L to about 320 mOsm/L or about 250 mOsm/L to about 320 mOsm/L.

In some embodiments, the deliverable osmolarity of any formulation described herein is designed to be isotonic with the targeted otic structure. In some embodiments, otic compositions described herein are formulated to provide a delivered osmolarity at the target site of action of about 250 to about 320 mOsm/L; and preferably about 270 to about 320 mOsm/L. In specific embodiments, otic compositions described herein are formulated to provide a delivered osmolality at the target site of action of about about 250 to about 320 mOsm/kg H₂O; or an osmolality of about 270 to about 320 mOsm/kg H₂O. In specific embodiments, the deliverable osmolarity/osmolality of the formulations (i.e., the osmolarity/osmolality of the formulation in the absence of gelling or thickening agents (e.g., thermoreversible gel polymers) is adjusted, for example, by the use of appropriate salt concentrations (e.g., concentration of potassium or sodium salts) or the use of tonicity agents which renders the formulations compatible upon delivery at the target site. The osmolarity of a formulation comprising a thermoreversible gel polymer is an unreliable measure due to the association of varying amounts of water with the monomeric units of the polymer. The practical osmolarity of a formulation (i.e., osmolarity in the absence of a gelling or thickening agent (e.g. a thermoreversible gel polymer) is a reliable measure and is measured by any suitable method (e.g., freezing point depression method, vapor depression method). In some instances, the formulations described herein provide a deliverable osmolarity (e.g., at a target site (e.g., EAC) that causes minimal disturbance to the environment and causes minimum discomfort (e.g., vertigo) to a mammal upon administration.

In some embodiments, suitable tonicity agents include, but are not limited to any pharmaceutically acceptable sugar, salt or any combinations or mixtures thereof, such as, but not limited to dextrose, glycerin, mannitol, sorbitol, sodium chloride, and other electrolytes.

Useful otic compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

### Particle Size

Size reduction is used to increase surface area and/or modulate formulation dissolution properties. It is also used to maintain a consistent average particle size distribution (PSD) (e.g., micrometer-sized particles, nanometer-sized particles or the like) for any formulation described herein. In some embodiments, any formulation described herein comprises multiparticulates, i.e., a plurality of particle sizes (e.g., micronized particles, nano-sized particles, non-sized particles, colloidal particles); i.e, the formulation is a multiparticulate formulation. In some embodiments, any formulation described herein comprises one or more multiparticulate (e.g., micronized) therapeutic agents. Micronization is a process of reducing the average diameter of particles of a solid material. Micronized particles are from about micrometer-sized in diameter to about nanometer-sized in diameter. In some embodiments, the average diameter of particles in a micronized solid is from about 0.5 μm to about 500 μm. In some embodiments, the average diameter of particles in a micronized solid is from about 1 μm to about 200 μm. In some embodiments, the average diameter of particles in a micronized solid is from about 2 μm to about 100 μm. In some embodiments, the average diameter of particles in a micronized solid is from about 3 μm to about 50 μm. In some embodiments, a particulate micronized solid comprises particle sizes of less than about 5 microns, less than about 20 microns and/or less than about 100 microns. In some embodiments, the use of particulates (e.g., micronized particles) of otic agent allows for extended and/or sustained release of the otic agent from any formulation described herein compared to a formulation comprising non-multiparticulate (e.g, non-micronized) otic agent. In some instances, formulations containing multiparticulate (e.g. micronized) otic agent are ejected from a 1 mL syringe adapted with a 27G needle without any plugging or clogging.

Particle size reduction techniques include, by way of example, grinding, milling (e.g., air-attrition milling (jet milling), ball milling), coacervation, complex coacervation, high pressure homogenization, spray drying and/or supercritical fluid crystallization. In some instances, particles are sized by mechanical impact (e.g., by hammer mills, ball mill and/or pin mills). In some instances, particles are sized via fluid energy (e.g., by spiral jet mills, loop jet mills, and/or fluidized bed jet mills). In some embodiments formulations described herein comprise crystalline particles and/or isotropic particles. In some embodiments, formulations described herein comprise amorphous particles and/or anisotropic particles. In some embodiments, formulations described herein comprise therapeutic agent particles wherein the therapeutic agent is a free base, or a salt, or a prodrug of a therapeutic agent, or any combination thereof.

In specific embodiments, any auris-compatible formulation described herein comprises one or more micronized pharmaceutical agents (e.g., otic agents). In some of such embodiments, a micronized pharmaceutical agent comprises micronized particles. In some of such embodiments, a micronized pharmaceutical agent comprising micronized particles of the pharmaceutical agent itself without any coating or encapsulation. In certain embodiments, a pharmaceutical composition described herein comprises an otic agent as a micronized powder. In certain embodiments, a pharmaceutical composition described herein comprises an otic agent in the form of a micronized otic agent powder.

In some embodiments, the multiparticulates and/or micronized otic agents described herein are delivered to an auris structure (e.g., EAC) by auris-acceptable gel matrices.

### Tuable Release Characteristics

The release of active agent from any formulation, or composition described herein is optionally tunable to the desired release characteristics. In some embodiments, a composition described herein is a solution comprising of gelling components. In some of such embodiments, the composition provides release of an active agent from about 1 days to about 14 days, about 1 days to about 12 days, about 2 days to about 10 days, or about 3 days to about 8 days.

In some embodiments, a composition described herein comprises a gelling agent (e.g., poloxamer 407) and provides release of an active agent over a period of from about 1 day to about 3 days. In some embodiments, a composition described herein comprises a gelling agent (e.g., poloxamer 407) and provides release of an active agent over a period of from about 1 day to about 5 days. In some embodiments, a composition described herein comprises a gelling agent (e.g., poloxamer 407) and provides release of an active agent over a period of from about 1 day to about 7 days. In some embodiments, a composition described herein comprises a gelling agent (e.g., poloxamer 407) and provides release of an active agent over a period of from about 2 days to about 7 days. In some embodiments, a composition described herein comprises a gelling agent (e.g., poloxamer 407) and provides release of an active agent over a period of from about 3 day to about 7 days. In some embodiments, a composition described herein comprises a gelling agent (e.g., poloxamer 407) and provides release of an active agent over a period of from about 1 day to about 10 days. In some embodiments, a composition described herein comprises a gelling agent (e.g., poloxamer 407) and provides release of an active agent over a period of from about 3 day to about 10 days. In some embodiments, a composition described herein comprises a gelling agent (e.g., poloxamer 407) and provides release of an active agent over a period of from about 1 day to about 14 days.

In some embodiments, a composition described herein comprises a gelling agent (e.g., poloxamer 407) in combination with micronized otic agent and provides extended sustained release over a longer period of time. In some embodiments, a composition described herein comprises about 10-25% of a gelling agent (e.g., poloxamer 407) and micronized otic agent, and provides extended sustained release over a period of from about 1 week to about 10 weeks. In some embodiments, a composition described herein comprises about 12-21% of a gelling agent (e.g., poloxamer 407) and micronized otic agent, and provides extended sustained release over a period of from about 1 week to about 6 weeks. In some embodiments, a composition described herein comprises about 14-17% of a gelling agent (e.g., poloxamer 407) and micronized otic agent, and provides extended sustained release over a period of from about 1 week to about 3 weeks. In some embodiments, a composition described herein comprises about 15-18% of a gelling agent (e.g., poloxamer 407) and micronized otic agent, and provides extended sustained release over a period of from about 1 week to about 3 weeks. In some embodiments, a composition described herein comprises about 18-21% of a gelling agent (e.g., poloxamer 407) and micronized otic agent, and provides extended sustained release over a period of from about 3 weeks to about 6 weeks.

Accordingly, the amount of gelling agent in a composition, and the particle size of an otic agent are tunable to the desired release profile of an otic agent from the composition.

As described herein, compositions comprising micronized otic agents provide extended release over a longer period of time compared to compositions comprising non-micronized otic agents. In some instances, the micronized otic agent provides a steady supply (e.g., +/−20%) of active agent via slow degradation and serves as a depot for the active agent; such a depot effect increases residence time of the otic agent in the ear. In specific embodiments, selection of an appropriate particle size of the active agent (e.g., micronized active agent) in combination with the amount of gelling agent in the composition provides tunable extended release characteristics that allow for release of an active agent over a period of hours, days, weeks or months.

In some embodiments, the viscosity of any formulation described herein is designed to provide a suitable rate of release from an auris compatible gel. In some embodiments, the concentration of a thickening agent (e.g., gelling components such as polyoxyethylene-polyoxypropylene copolymers) allows for a tunable mean dissolution time (MDT). The MDT is inversely proportional to the release rate of an active agent from a composition described herein. Experimentally, the released otic agent is optionally fitted to the Korsmeyer-Peppas equation

$$\frac{Q}{{Q}_{α}} = {kt}^{n} + b$$

where Q is the amount of otic agent released at time t, Qα is the overall released amount of otic agent, k is a release constant of the nth order, n is a dimensionless number related to the dissolution mechanism and b is the axis intercept, characterizing the initial burst release mechanism wherein n=1 characterizes an erosion controlled mechanism. The mean dissolution time (MDT) is the sum of different periods of time the drug molecules stay in the matrix before release, divided by the total number of molecules and is optionally calculated by:

$$MDT = \frac{{nk}^{- 1 / n}}{n + 1}$$

For example, a linear relationship between the mean dissolution time (MDT) of a composition and the concentration of the gelling agent (e.g., poloxamer) indicates that the otic agent is released due to the erosion of the polymer gel (e.g., poloxamer) and not via diffusion. In another example, a non-linear relationship indicates release of otic agent via a combination of diffusion and/or polymer gel degradation. In another example, a faster gel elimination time course of a composition (a faster release of active agent) indicates lower mean dissolution time (MDT). The concentration of gelling components and/or active agent in a composition are tested to determine suitable parameters for MDT. In some embodiments, injection volumes are also tested to determine suitable parameters for preclinical and clinical studies. The gel strength and concentration of the active agent affects release kinetics of an otic agent from the composition. At low poloxamer concentration, elimination rate is accelerated (MDT is lower). An increase in otic agent concentration in the composition prolongs residence time and/or MDT of the otic agent in the ear.

In some embodiments, the MDT for poloxamer from a composition described herein is at least 6 hours. In some embodiments, the MDT for poloxamer from a composition described herein is at least 10 hours.

In some embodiments, the MDT for an active agent from a composition described herein is from about 30 hours to about 48 hours. In some embodiments, the MDT for an active agent from a composition described herein is from about 30 hours to about 96 hours. In some embodiments, the MDT for an active agent from a composition described herein is from about 30 hours to about 1 week. In some embodiments, the MDT for a composition described herein is from about 1 week to about 6 weeks.

In some embodiments, the mean residence time (MRT) for an active agent in a composition described herein is from about 20 hours to about 48 hours. In some embodiments, the MRT for an active agent from a composition described herein is from about 20 hours to about 96 hours. In some embodiments, the MRT for an active agent from a composition described herein is from about 20 hours to about 1 week.

In some embodiments, the MRT for an active agent is about 20 hours. In some embodiments, the MRT for an active agent is about 30 hours. In some embodiments, the MRT for an active agent is about 40 hours. In some embodiments, the MRT for an active agent is about 50 hours. In some embodiments, the MRT for an active agent is about 60 hours. In some embodiments, the MRT for an active agent is about 70 hours. In some embodiments, the MRT for an active agent is about 80 hours. In some embodiments, the MRT for an active agent is about 90 hours. In some embodiments, the MRT for an active agent is about 1 week. In some embodiments, the MRT for an active agent is about 90 hours. In some embodiments, the MRT for a composition described herein is from about 1 week to about 6 weeks. In some embodiments, the MRT for an active agent is about 1 week. In some embodiments, the MRT for an active agent is about 2 weeks. In some embodiments, the MRT for an active agent is about 3 weeks. In some embodiments, the MRT for an active agent is about 4 weeks. In some embodiments, the MRT for an active agent is about 5 weeks. In some embodiments, the MRT for an active agent is about 4 weeks. In some embodiments, the MRT for an active agent is about 6 weeks. The half life of an otic agent and mean residence time of the otic agent are determined for each formulation by measurement of concentration of the otic agent in the EAC using procedures described herein.

In certain embodiments, any controlled release otic formulation described herein increases the exposure of an otic agent and increases the Area Under the Curve (AUC) in the EAC by about 30%, about 40%, about 50%, about 60%, about 70%, about 80% or about 90% compared to a formulation that is not a controlled release otic formulation. In certain embodiments, any controlled release otic formulation described herein increases the exposure time of an otic agent and decreases the Cₘₐₓ in the EAC by about 40%, about 30%, about 20%, or about 10%, compared to a formulation that is not a controlled release otic formulation. In certain embodiments, any controlled release otic formulation described herein alters (e.g. reduces) the ratio of Cₘₐₓ to Cₘᵢₙ compared to a formulation that is not a controlled release otic formulation. In certain embodiments, any controlled release otic formulation described herein increases the exposure of an otic agent and increases the length of time that the concentration of an otic agent is above Cₘᵢₙ by about 30%, about 40%, about 50%, about 60%, about 70%, about 80% or about 90% compared to a formulation that is not a controlled release otic formulation. In certain instances, controlled release formulations described herein delay the time to Cₘₐₓ. In certain instances, the controlled steady release of a drug prolongs the time the concentration of the drug will stay above the Cₘᵢₙ. In some embodiments, otic compositions described herein prolong the residence time of a drug in the ear and provide a stable drug exposure profile. In some instances, an increase in concentration of an active agent in the composition saturates the clearance process and allows for a more rapid and stable steady state to be reached.

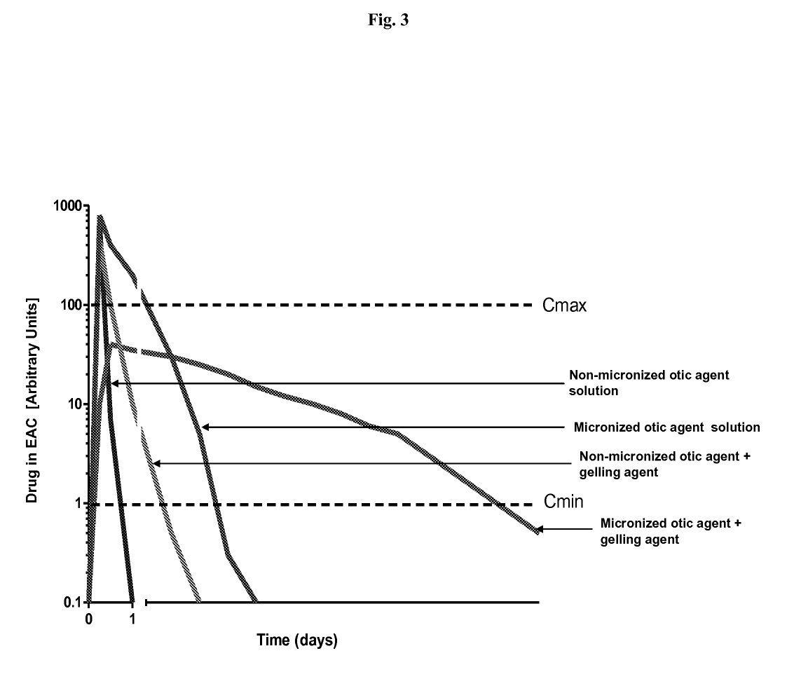

In certain instances, once drug exposure (e.g. EAC) of a drug reaches steady state, the concentration of the drug in the EAC stays at or about the therapeutic dose for an extended period of time (e.g., at least one day, at least 2 days, at least 3 days, at least 4 days, at least 5 days, at least 6 days, at least 1 week, at least 3 weeks, at least 6 weeks, or at least 2 months). In some embodiments, the steady state concentration of active agent released from a controlled release formulation described herein is about 5 to about 20 times the steady state concentration of an active agent released from a formulation that is not a controlled release formulation. In some embodiments, the steady state concentration of active agent released from a controlled release formulation described herein is about 20 to about 50 times the steady state concentration of an active agent released from a formulation that is not a controlled release formulation. FIG. 3 illustrates predicted tunable release of an active agent from four compositions.

### Pharmaceutical Formulations

Provided herein are pharmaceutical compositions that include at least one otic agent and a pharmaceutically acceptable diluent(s), excipient(s), or carrier(s). In some embodiments, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers. In other embodiments, the pharmaceutical compositions also contain other therapeutic substances.

In some embodiments, the compositions described herein include a dye to help enhance the visualization of the gel when applied to the EAC. In some embodiments, dyes that are compatible with the auris-acceptable compositions described herein include Evans blue (e.g., 0.5% of the total weight of an otic formulation), Methylene blue (e.g., 1% of the total weight of an otic formulation), Isosulfan blue (e.g., 1% of the total weight of an otic formulation), Trypan blue (e.g., 0.15% of the total weight of an otic formulation), and/or indocyanine green (e.g., 25 mg/vial). Other common dyes, e.g, FD&C red 40, FD&C red 3, FD&C yellow 5, FD&C yellow 6, FD&C blue 1, FD&C blue2, FD&C green 3, fluorescence dyes (e.g., Fluorescein isothiocyanate, rhodamine, Alexa Fluors, DyLight Fluors) and/or dyes that are visualizable in conjunction with non-invasive imaging techniques such as MRI, CAT scans, PET scans or the like. Gadolinium-based MRI dyes, iodine-base dyes, barium-based dyes or the like are also contemplated for use with any otic formulation described herein. Other dyes that are compatible with any formulation or composition described herein are listed in the Sigma-Aldrich catalog under dyes (which is included herein by reference for such disclosure).

In some embodiments, in the auris-acceptable controlled release otic formulations described herein, the otic agent is provided in a gel matrix, also referred to herein as “auris acceptable gel formulations,” “auris externa-acceptable gel formulations”, “auris gel formulations” or variations thereof. All of the components of the gel formulation must be compatible with the targeted auris structure. Further, the gel formulations provide controlled release of the otic agent to the desired site within the targeted auris structure; in some embodiments, the gel formulation also has an immediate or rapid release component for delivery of the otic agent to the desired target site. In other embodiments, the gel formulation has a sustained release component for delivery of the otic agent. In some embodiments, the gel formulation comprises a multiparticulate (e.g., micronized) otic agent. In some embodiments, the auris gel formulations are biodegradable. In some embodiments, the auris gel formulations are bioerodable.

In some embodiments, the auris gel formulation contains a viscosity enhancing agent sufficient to provide a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise. In some embodiments, the auris gel formulation contains a viscosity enhancing agent sufficient to provide a viscosity of between about 50,0000 and 1,000,000 centipoise.

In some embodiments, the compositions described herein are low viscosity compositions at body temperature. In some embodiments, low viscosity compositions contain from about 1% to about 10% of a viscosity enhancing agent (e.g., gelling components such as polyoxyethylene-polyoxypropylene copolymers). In some embodiments, low viscosity compositions contain from about 2% to about 10% of a viscosity enhancing agent (e.g., gelling components such as polyoxyethylene-polyoxypropylene copolymers). In some embodiments, low viscosity compositions contain from about 5% to about 10% of a viscosity enhancing agent (e.g., gelling components such as polyoxyethylene-polyoxypropylene copolymers). In some embodiments, low viscosity compositions are substantially free of a viscosity enhancing agent (e.g., gelling components such as polyoxyethylene-polyoxypropylene copolymers). In some embodiments, a low viscosity otic agent composition described herein provides an apparent viscosity of from about 100 cP to about 10,000 cP. In some embodiments, a low viscosity otic agent composition described herein provides an apparent viscosity of from about 500 cP to about 10,000 cP. In some embodiments, a low viscosity otic agent composition described herein provides an apparent viscosity of from about 1000 cP to about 10,000 cP.

In some embodiments, the compositions described herein are high viscosity compositions at body temperature. In some embodiments, high viscosity compositions contain from about 10% to about 25% of a viscosity enhancing agent (e.g., gelling components such as polyoxyethylene-polyoxypropylene copolymers). In some embodiments, high viscosity compositions contain from about 14% to about 22% of a viscosity enhancing agent (e.g., gelling components such as polyoxyethylene-polyoxypropylene copolymers). In some embodiments, high viscosity compositions contain from about 15% to about 21% of a viscosity enhancing agent (e.g., gelling components such as polyoxyethylene-polyoxypropylene copolymers). In some embodiments, a high viscosity otic agent composition described herein provides an apparent viscosity of from about 100,000 cP to about 1,000,000 cP. In some embodiments, a high viscosity otic agent composition described herein provides an apparent viscosity of from about 150,000 cP to about 500,000 cP. In some embodiments, a high viscosity otic agent composition described herein provides an apparent viscosity of from about 250,000 cP to about 500,000 cP. In some of such embodiments, a high viscosity composition is a liquid at room temperature and gels at about between room temperature and body temperature (including an individual with a serious fever, e.g., up to about 42° C.). In some embodiments, an otic agent high viscosity composition is administered as monotherapy for treatment of an otic disease or condition described herein.

In some embodiments, the otic pharmaceutical formulations described herein further provide an auris-acceptable hydrogel; in still further embodiments, the auris pharmaceutical formulations provide an auris-acceptable in situ forming hydrogel material. In some embodiments, the auris pharmaceutical formulations provide an auris-acceptable solvent release gel. In some embodiments, the auris pharmaceutical formulations provide an actinic radiation curable gel. Further embodiments include a thermoreversible gel in the auris pharmaceutical formulation, such that upon preparation of the gel at room temperature or below, the formulation is a fluid, but upon application of the gel into or near the EAC target site, including the outer surface of the tympanic membrane, the auris-pharmaceutical formulation stiffens or hardens into a gel-like substance.

In some embodiments, the auris gel formulations are capable of being administered on or near the outer surface of the tympanic membrane via syringe and needle. In some embodiments, the auris gel formulations are capable of being administered on or near the outer surface of the tympanic membrane via a syringe. In some embodiments, the auris gel formulations are capable of being administered on or near the outer surface of the tympanic membrane via a dropper. In other embodiments, the auris gel formulations are administered onto the external auditory canal. In some embodiments, the formulations are administered via a pump device or another device capable of delivering the formulations onto or near the outer surface of the tympanic membrane, onto the external auditory canal, or a combination thereof.

In some embodiments, any pharmaceutical composition described herein comprises a multiparticulate otic agent in a liquid matrix (e.g., a liquid composition for injection, or otic drops). In certain embodiments, any pharmaceutical composition described herein comprises a multiparticulate otic agent in a solid matrix.

### Controlled Release Formulations

In general, controlled release drug formulations impart control over the release of drug with respect to site of release and time of release within the body. As discussed herein, controlled release refers to immediate release, delayed release, sustained release, extended release, variable release, pulsatile release and bi-modal release. Many advantages are offered by controlled release. First, controlled release of a pharmaceutical agent allows less frequent dosing and thus minimizes repeated treatment. Second, controlled release treatment results in more efficient drug utilization and less of the compound remains as a residue. Third, controlled release offers the possibility of localized drug delivery by placement of a delivery device or formulation at the site of disease. Still further, controlled release offers the opportunity to administer and release two or more different drugs, each having a unique release profile, or to release the same drug at different rates or for different durations, by means of a single dosage unit.

Accordingly, one aspect of the embodiments disclosed herein is to provide a controlled release auris-acceptable composition for modulating the production of cerumen and the treatment of ceruminosis and ceruminosis associated diseases. The controlled release aspect of the compositions and/or formulations and/or devices disclosed herein is imparted through a variety of agents, including but not limited to excipients, agents or materials that are acceptable for use in the EAC. By way of example only, such excipients, agents or materials include an auris-acceptable polymer, an auris-acceptable viscosity enhancing agent, an auris-acceptable gel, an auris-acceptable hydrogel, an auris-acceptable in situ forming hydrogel material, an auris-acceptable actinic radiation curable gel, an auris-acceptable solvent release gel, an auris-acceptable nanocapsule or nanosphere, an auris-acceptable thermoreversible gel, or combinations thereof.

### Auris-Acceptable Gels

Gels, sometimes referred to as jellies, have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

Gels can also be classified as being hydrophobic or hydrophilic. In certain embodiments, the base of a hydrophobic gel consists of a liquid paraffin with polyethylene or fatty oils gelled with colloidal silica, or aluminum or zinc soaps. In contrast, the base of hydrophobic gels usually consists of water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions disclosed herein is pseudo plastic, plastic, thixotropic, or dilatant.

In one embodiment the enhanced viscosity auris-acceptable formulation described herein is not a liquid at room temperature. In certain embodiments, the enhanced viscosity formulation is characterized by a phase transition between room temperature and body temperature (including an individual with a serious fever, e.g., up to about 42° C.). In some embodiments, the phase transition occurs at 1° C. below body temperature, at 2° C. below body temperature, at 3° C. below body temperature, at 4° C. below body temperature, at 6° C. below body temperature, at 8° C. below body temperature, or at 10° C. below body temperature. In some embodiments, the phase transition occurs at about 15° C. below body temperature, at about 20° C. below body temperature or at about 25° C. below body temperature. In specific embodiments, the gelation temperature (Tgel) of a formulation described herein is about 20° C., about 25° C., or about 30° C. In certain embodiments, the gelation temperature (Tgel) of a formulation described herein is about 35° C., or about 40° C. Included within the definition of body temperature is the body temperature of a healthy individual, or an unhealthy individual, including an individual with a fever (up to ˜42° C.). In some embodiments, the pharmaceutical compositions described herein are liquids at about room temperature and are administered at or about room temperature.

Polymers composed of polyoxypropylene and polyoxyethylene form thermoreversible gels when incorporated into aqueous solutions. These polymers have the ability to change from the liquid state to the gel state at temperatures close to body temperature, therefore allowing useful formulations that are applied to the targeted auris structure(s). The liquid state-to-gel state phase transition is dependent on the polymer concentration and the ingredients in the solution.

In some embodiments, the amount of thermoreversible polymer in any formulation described herein is about 10%, about 15%, about 20%, about 25%, about 30%, about 35% or about 40% of the total weight of the formulation. In some embodiments, the amount of thermoreversible polymer in any formulation described herein is about 10%, about 11%, about 12%, about 13%, about 14%, about 15%, about 16%, about 17%, about 18%, about 19%, about 20%, about 21%, about 22%, about 23%, about 24% or about 25% of the total weight of the formulation. In some embodiments, the amount of thermoreversible polymer (e.g., Poloxamer 407) in any formulation described herein is about 7.5% of the total weight of the formulation. In some embodiments, the amount of thermoreversible polymer (e.g., Poloxamer 407) in any formulation described herein is about 10% of the total weight of the formulation. In some embodiments, the amount of thermoreversible polymer (e.g., Poloxamer 407) in any formulation described herein is about 11% of the total weight of the formulation. In some embodiments, the amount of thermoreversible polymer (e.g., Poloxamer 407) in any formulation described herein is about 12% of the total weight of the formulation. In some embodiments, the amount of thermoreversible polymer (e.g., Poloxamer 407) in any formulation described herein is about 13% of the total weight of the formulation. In some embodiments, the amount of thermoreversible polymer (e.g., Poloxamer 407) in any formulation described herein is about 14% of the total weight of the formulation. In some embodiments, the amount of thermoreversible polymer (e.g., Poloxamer 407) in any formulation described herein is about 15% of the total weight of the formulation. In some embodiments, the amount of thermoreversible polymer (e.g., Poloxamer 407) in any formulation described herein is about 16% of the total weight of the formulation. In some embodiments, the amount of thermoreversible polymer (e.g., Poloxamer 407) in any formulation described herein is about 17% of the total weight of the formulation. In some embodiments, the amount of thermoreversible polymer (e.g., Poloxamer 407) in any formulation described herein is about 18% of the total weight of the formulation. In some embodiments, the amount of thermoreversible polymer (e.g., Poloxamer 407) in any formulation described herein is about 19% of the total weight of the formulation. In some embodiments, the amount of thermoreversible polymer (e.g., Poloxamer 407) in any formulation described herein is about 20% of the total weight of the formulation. In some embodiments, the amount of thermoreversible polymer (e.g., Poloxamer 407) in any formulation described herein is about 21% of the total weight of the formulation. In some embodiments, the amount of thermoreversible polymer (e.g., Poloxamer 407) in any formulation described herein is about 23% of the total weight of the formulation. In some embodiments, the amount of thermoreversible polymer (e.g., Poloxamer 407) in any formulation described herein is about 25% of the total weight of the formulation. In some embodiments, the amount of thickening agent (e.g., a gelling agent) in any formulation described herein is about 1%, about 5%, about 10%, or about 15% of the total weight of the formulation. In some embodiments, the amount of thickening agent (e.g., a gelling agent) in any formulation described herein is about 0.5%, about 1%, about 1.5%, about 2%, about 2.5%, about 3%, about 3.5%, about 4%, about 4.5%, or about 5% of the total weight of the formulation.

In an alternative embodiment, the thermogel is a PEG-PLGA-PEG triblock copolymer (Jeong et al, Nature (1997), 388:860-2; Jeong et al, J. Control. Release (2000), 63:155-63; Jeong et al, Adv. Drug Delivery Rev. (2002), 54:37-51). The polymer exhibits sol-gel behavior over a concentration of about 5% w/w to about 40% w/w. Depending on the properties desired, the lactide/glycolide molar ratio in the PLGA copolymer ranges from about 1:1 to about 20:1. The resulting coploymers are soluble in water and form a free-flowing liquid at room temperature, but form a hydrogel at body temperature. A commercially available PEG-PLGA-PEG triblock copolymer is RESOMER RGP t50106 manufactured by Boehringer Ingelheim. This material is composed of a PLGA copolymer of 50:50 poly(DL-lactide-co-glycolide) and is 10% w/w of PEG and has a molecular weight of about 6000.

Additional biodegradable thermoplastic polyesters include AtriGel® (provided by Atrix Laboratories, Inc.) and/or those disclosed, e.g., in U.S. Pat. Nos. 5,324,519; 4,938,763; 5,702,716; 5,744,153; and 5,990,194; wherein the suitable biodegradable thermoplastic polyester is disclosed as a thermoplastic polymer. Examples of suitable biodegradable thermoplastic polyesters include polylactides, polyglycolides, polycaprolactones, copolymers thereof, terpolymers thereof, and any combinations thereof. In some such embodiments, the suitable biodegradable thermoplastic polyester is a polylactide, a polyglycolide, a copolymer thereof, a terpolymer thereof, or a combination thereof. In one embodiment, the biodegradable thermoplastic polyester is 50/50 poly(DL-lactide-co-glycolide) having a carboxy terminal group; is present in about 30 wt. % to about 40 wt. % of the composition; and has an average molecular weight of about 23,000 to about 45,000. Alternatively, in another embodiment, the biodegradable thermoplastic polyester is 75/25 poly (DL-lactide-co-glycolide) without a carboxy terminal group; is present in about 40 wt. % to about 50 wt. % of the composition; and has an average molecular weight of about 15,000 to about 24,000. In further or alternative embodiments, the terminal groups of the poly(DL-lactide-co-glycolide) are either hydroxyl, carboxyl, or ester depending upon the method of polymerization. Polycondensation of lactic or glycolic acid provides a polymer with terminal hydroxyl and carboxyl groups. Ring-opening polymerization of the cyclic lactide or glycolide monomers with water, lactic acid, or glycolic acid provides polymers with the same terminal groups. However, ring-opening of the cyclic monomers with a monofunctional alcohol such as methanol, ethanol, or 1-dodecanol provides a polymer with one hydroxyl group and one ester terminal groups. Ring-opening polymerization of the cyclic monomers with a diol such as 1,6-hexanediol or polyethylene glycol provides a polymer with only hydroxyl terminal groups.

Since the polymer systems of thermoreversible gels dissolve more completely at reduced temperatures, methods of solubilization include adding the required amount of polymer to the amount of water to be used at reduced temperatures. Generally after wetting the polymer by shaking, the mixture is capped and placed in a cold chamber or in a thermostatic container at about 0-10° C. in order to dissolve the polymer. The mixture is stirred or shaken to bring about a more rapid dissolution of the thermoreversible gel polymer. The otic agent and various additives such as buffers, salts, and preservatives are subsequently added and dissolved. In some instances the otic agent and/or other pharmaceutically active agent is suspended if it is insoluble in water. The pH is modulated by the addition of appropriate buffering agents.

In one embodiment are auris-acceptable pharmaceutical gel formulations which do not require the use of an added viscosity enhancing agent. Such gel formulations incorporate at least one pharmaceutically acceptable buffer. In one aspect is a gel formulation comprising an otic agent and a pharmaceutically acceptable buffer. In another embodiment, the pharmaceutically acceptable excipient or carrier is a gelling agent.

In other embodiments, useful otic agent auris-acceptable pharmaceutical formulations also include one or more pH adjusting agents or buffering agents to provide an EAC suitable pH. Suitable pH adjusting agents or buffers include, but are not limited to acetate, bicarbonate, ammonium chloride, citrate, phosphate, pharmaceutically acceptable salts thereof and combinations or mixtures thereof. Such pH adjusting agents and buffers are included in an amount required to maintain pH of the composition between a pH of about 5 and about 9, in one embodiment a pH between about 6.5 to about 7.5, and in yet another embodiment at a pH of about 6.5, 6.6, 6.7, 6.8, 6.9, 7.0, 7.1, 7.2, 7.3, 7.4, 7.5. In one embodiment, when one or more buffers are utilized in the formulations of the present disclosure, they are combined, e.g., with a pharmaceutically acceptable vehicle and are present in the final formulation, e.g., in an amount ranging from about 0.1% to about 20%, from about 0.5% to about 10%. In certain embodiments of the present disclosure, the amount of buffer included in the gel formulations are an amount such that the pH of the gel formulation does not interfere with the EAC's natural buffering system. In some embodiments, from about 10 μM to about 200 mM concentration of a buffer is present in the gel formulation. In certain embodiments, from about a 5 mM to about a 200 mM concentration of a buffer is present. In certain embodiments, from about a 20 mM to about a 100 mM concentration of a buffer is present. In one embodiment is a buffer such as acetate or citrate at slightly acidic pH. In one embodiment the buffer is a sodium acetate buffer having a pH of about 4.5 to about 6.5. In one embodiment the buffer is a sodium citrate buffer having a pH of about 5.0 to about 8.0, or about 5.5 to about 7.0.

In an alternative embodiment, the buffer used is tris(hydroxymethyl)aminomethane, bicarbonate, carbonate or phosphate at slightly basic pH. In one embodiment, the buffer is a sodium bicarbonate buffer having a pH of about 6.5 to about 8.5, or about 7.0 to about 8.0. In another embodiment the buffer is a sodium phosphate dibasic buffer having a pH of about 6.0 to about 9.0.

Also described herein are controlled release formulations comprising an otic agent and a viscosity enhancing agent. Suitable viscosity-enhancing agents include by way of example only, gelling agents and suspending agents. In one embodiment, the enhanced viscosity formulation does not include a buffer. In other embodiments, the enhanced viscosity formulation includes a pharmaceutically acceptable buffer. Sodium chloride or other tonicity agents are optionally used to adjust tonicity, if necessary.

By way of example only, the auris-acceptable viscosity agent includes hydroxypropyl methylcellulose, hydroxyethyl cellulose, polyvinylpyrrolidone, carboxymethyl cellulose, polyvinyl alcohol, sodium chondroitin sulfate, sodium hyaluronate. Other viscosity enhancing agents compatible with the targeted auris structure include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose) or combinations thereof. In specific embodiments, the viscosity-enhancing excipient is a combination of MCC and CMC. In another embodiment, the viscosity-enhancing agent is a combination of carboxymethylated chitosan, or chitin, and alginate. The combination of chitin and alginate with the otic agents disclosed herein acts as a controlled release formulation, restricting the diffusion of the otic agents from the formulation. Moreover, the combination of carboxymethylated chitosan and alginate is optionally used to assist in increasing the permeability of the otic agents through the skin of the EAC.

In some embodiments is an enhanced viscosity formulation, comprising from about 0.1 mM and about 100 mM of an otic agent, a pharmaceutically acceptable viscosity agent, and water for injection, the concentration of the viscosity agent in the water being sufficient to provide a enhanced viscosity formulation with a final viscosity from about 100 to about 100,000 cP. In certain embodiments, the viscosity of the gel is in the range from about 100 to about 50,000 cP, about 100 cP to about 1,000 cP, about 500 cP to about 1500 cP, about 1000 cP to about 3000 cP, about 2000 cP to about 8,000 cP, about 4,000 cP to about 50,000 cP, about 10,000 cP to about 500,000 cP, about 15,000 cP to about 1,000,000 cP. In other embodiments, when an even more viscous medium is desired, the biocompatible gel comprises at least about 35%, at least about 45%, at least about 55%, at least about 65%, at least about 70%, at least about 75%, or even at least about 80% or so by weight of the otic agent. In highly concentrated samples, the biocompatible enhanced viscosity formulation comprises at least about 25%, at least about 35%, at least about 45%, at least about 55%, at least about 65%, at least about 75%, at least about 85%, at least about 90% or at least about 95% or more by weight of the otic agent.

In some embodiments, the viscosity of the gel formulations presented herein is measured by any means described. For example, in some embodiments, an LVDV-II+CP Cone Plate Viscometer and a Cone Spindle CPE-40 is used to calculate the viscosity of the gel formulation described herein. In other embodiments, a Brookfield (spindle and cup) viscometer is used to calculate the viscosity of the gel formulation described herein. In some embodiments, the viscosity ranges referred to herein are measured at room temperature. In other embodiments, the viscosity ranges referred to herein are measured at body temperature (e.g., at the average body temperature of a healthy human).

In one embodiment, the pharmaceutically acceptable enhanced viscosity auris-acceptable formulation comprises at least one otic agent and at least one gelling agent. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof. In some other embodiments, hydroxypropylmethylcellulose (Methocel®) is utilized as the gelling agent. In certain embodiments, the viscosity enhancing agents described herein are also utilized as the gelling agent for the gel formulations presented herein.

In some embodiments, other gel formulations are useful depending upon the particular otic agent, other pharmaceutical agent or excipients/additives used, and as such are considered to fall within the scope of the present disclosure. For example, other commercially-available glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the otic agent formulations described herein. In some embodiments, auris-acceptable gels include, but are not limited to, alginate hydrogels SAF®-Gel (ConvaTec, Princeton, N.J.), Duoderm® Hydroactive Gel (ConvaTec), Nu-gel®(Johnson & Johnson Medical, Arlington, Tex.); Carrasyn®(V) Acemannan Hydrogel (Carrington Laboratories, Inc., Irving, Tex.); glycerin gels Elta® Hydrogel (Swiss-American Products, Inc., Dallas, Tex.) and K-Y® Sterile (Johnson & Johnson). In further embodiments, biodegradable biocompatible gels also represent compounds present in auris-acceptable formulations disclosed and described herein.

In some formulations developed for administration to a mammal, and for compositions formulated for human administration, the auris-acceptable gel comprises substantially all of the weight of the composition. In other embodiments, the auris-acceptable gel comprises as much as about 98% or about 99% of the composition by weight. This is desirous when a substantially non-fluid, or substantially viscous formulation is needed. In a further embodiment, when slightly less viscous, or slightly more fluid auris-acceptable pharmaceutical gel formulations are desired, the biocompatible gel portion of the formulation comprises at least about 50% by weight, at least about 60% by weight, at least about 70% by weight, or even at least about 80% or 90% by weight of the compound. All intermediate integers within these ranges are contemplated to fall within the scope of this disclosure, and in some alternative embodiments, even more fluid (and consequently less viscous) auris-acceptable gel compositions are formulated, such as for example, those in which the gel or matrix component of the mixture comprises not more than about 50% by weight, not more than about 40% by weight, not more than about 30% by weight, or even those than comprise not more than about 15% or about 20% by weight of the composition.

Auris-Acceptable Suspending Agents

In one embodiment, at least one otic agent is included in a pharmaceutically acceptable enhanced viscosity formulation wherein the formulation further comprises at least one suspending agent, wherein the suspending agent assists in imparting controlled release characteristics to the formulation. In some embodiments, suspending agents also serve to increase the viscosity of the auris-acceptable formulations and compositions.

Suspending agents include, by way of example only, compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), sodium carboxymethylcellulose, methylcellulose, hydroxypropylmethylcellulose (hypromellose), hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like. In some embodiments, useful aqueous suspensions also contain one or more polymers as suspending agents. Useful polymers include water-soluble polymers such as cellulosic polymers, e.g., hydroxypropyl methylcellulose, and water-insoluble polymers such as crosslinked carboxyl-containing polymers.

In one embodiment, the present disclosure provides auris-acceptable gel compositions comprising a therapeutically effective amount of an otic agent in a hydroxyethyl cellulose gel. Hydroxyethyl cellulose (HEC) is obtained as a dry powder which is reconstituted in water or an aqueous buffer solution to give the desired viscosity (generally about 200 cps to about 30,000 cps, corresponding to about 0.2 to about 10% HEC). In one embodiment the concentration of HEC is between about 1% and about 15%, about 1% and about 2%, or about 1.5% to about 2%.

In other embodiments, the auris-acceptable formulations, including gel formulations and viscosity-enhanced formulations, further include excipients, other medicinal or pharmaceutical agents, carriers, adjuvants, such as preserving, stabilizing, wetting or emulsifying agents, solution promoters, salts, solubilizers, an antifoaming agent, an antioxidant, a dispersing agent, a wetting agent, a surfactant, and combinations thereof.

Auris-Acceptable Actinic Radiation Curable Gel

In other embodiments, the gel is an actinic radiation curable gel, such that following administration to or near the targeted auris structure, use of actinic radiation (or light, including UV light, visible light, or infrared light) the desired gel properties are formed. By way of example only, fiber optics are used to provide the actinic radiation so as to form the desired gel properties. In some embodiments, the fiber optics and the gel administration device form a single unit. In other embodiments, the fiber optics and the gel administration device are provided separately.

Auris-Acceptable Solvent Release Gel

In some embodiments, the gel is a solvent release gel such that the desired gel properties are formed after administration to or near the targeted auris structure, that is, as the solvent in the injected gel formulation diffuses out the gel, a gel having the desired gel properties is formed. For example, a formulation that comprises sucrose acetate isobutyrate, a pharmaceutically acceptable solvent, one or more additives, and the otic agent is administered within the EAC: diffusion of the solvent out of the injected formulation provides a depot having the desired gel properties. For example, use of a water soluble solvent provides a high viscosity depot when the solvent diffuses rapidly out of the injected formulation. On the other hand, use of a hydrophobic solvent (e.g., benzyl benzoate) provides a less viscous depot. One example of an auris-acceptable solvent release gel formulation is the SABER™ Delivery System marketed by DURECT Corporation.

Auris-Acceptable Cyclodextrin and Other Stabilizing Formulations

In a specific embodiment, the auris-acceptable formulations alternatively comprises a cyclodextrin. Cyclodextrins are cyclic oligosaccharides containing 6, 7, or 8 glucopyranose units, referred to as α-cyclodextrin, β-cyclodextrin, or γ-cyclodextrin respectively. Cyclodextrins have a hydrophilic exterior, which enhances water-soluble, and a hydrophobic interior which forms a cavity. In an aqueous environment, hydrophobic portions of other molecules often enter the hydrophobic cavity of cyclodextrin to form inclusion compounds. Additionally, cyclodextrins are also capable of other types of nonbonding interactions with molecules that are not inside the hydrophobic cavity. Cyclodextrins have three free hydroxyl groups for each glucopyranose unit, or 18 hydroxyl groups on α-cyclodextrin, 21 hydroxyl groups on β-cyclodextrin, and 24 hydroxyl groups on γ-cyclodextrin. One or more of these hydroxyl groups can be reacted with any of a number of reagents to form a large variety of cyclodextrin derivatives, including hydroxypropyl ethers, sulfonates, and sulfoalkylethers. Shown below is the structure of β-cyclodextrin and the hydroxypropyl-β-cyclodextrin (HPβCD).

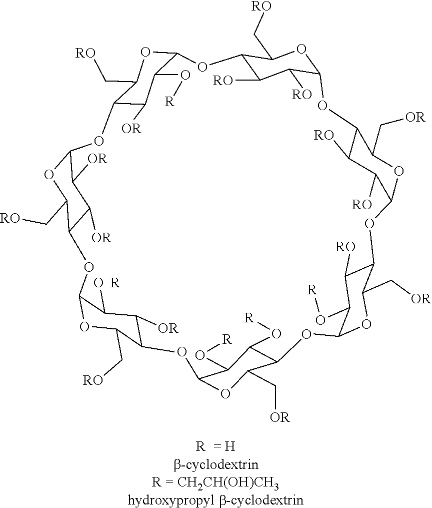

In some embodiments, the use of cyclodextrins in the pharmaceutical compositions described herein improves the solubility of the drug. Inclusion compounds are involved in many cases of enhanced solubility; however other interactions between cyclodextrins and insoluble compounds also improves solubility. Hydroxypropyl-β-cyclodextrin (HPβCD) is commercially available as a pyrogen free product. It is a nonhygroscopic white powder that readily dissolves in water. HPβCD is thermally stable and does not degrade at neutral pH. Thus, cyclodextrins improve the solubility of a therapeutic agent in a composition or formulation. Accordingly, in some embodiments, cyclodextrins are included to increase the solubility of the auris-acceptable otic agents within the formulations described herein. In other embodiments, cyclodextrins in addition serve as controlled release excipients within the formulations described herein.

By way of example only, cyclodextrin derivatives for use include α-cyclodextrin, β-cyclodextrin, γ-cyclodextrin, hydroxyethyl β-cyclodextrin, hydroxypropyl γ-cyclodextrin, sulfated β-cyclodextrin, sulfated α-cyclodextrin, sulfobutyl ether β-cyclodextrin.

The concentration of the cyclodextrin used in the compositions and methods disclosed herein varies according to the physiochemical properties, pharmacokinetic properties, side effect or adverse events, formulation considerations, or other factors associated with the therapeutically active agent, or a salt or prodrug thereof, or with the properties of other excipients in the composition. Thus, in certain circumstances, the concentration or amount of cyclodextrin used in accordance with the compositions and methods disclosed herein will vary, depending on the need. When used, the amount of cyclodextrins needed to increase solubility of the otic agent and/or function as a controlled release excipient in any of the formulations described herein is selected using the principles, examples, and teachings described herein.

Other stabilizers that are useful in the auris-acceptable formulations disclosed herein include, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used. In further embodiments, the chosen stabilizer changes the hydrophobicity of the formulation (e.g., oleic acid, waxes), or improves the mixing of various components in the formulation (e.g., ethanol), controls the moisture level in the formula (e.g., PVP or polyvinyl pyrrolidone), controls the mobility of the phase (substances with melting points higher than room temperature such as long chain fatty acids, alcohols, esters, ethers, amides etc. or mixtures thereof; waxes). In another embodiment some of these stabilizers are used as solvents/co-solvents (e.g., ethanol). In other embodiments, stabilizers are present in sufficient amounts to inhibit the degradation of the otic agent. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

Additional useful otic agent auris-acceptable formulations include one or more anti-aggregation additives to enhance stability of otic agent formulations by reducing the rate of protein aggregation. The anti-aggregation additive selected depends upon the nature of the conditions to which the otic agents, for example otic agent antibodies are exposed. For example, certain formulations undergoing agitation and thermal stress require a different anti-aggregation additive than a formulation undergoing lyophilization and reconstitution. Useful anti-aggregation additives include, by way of example only, urea, guanidinium chloride, simple amino acids such as glycine or arginine, sugars, polyalcohols, polysorbates, polymers such as polyethylene glycol and dextrans, alkyl saccharides, such as alkyl glycoside, and surfactants.

Other useful formulations optionally include one or more auris-acceptable antioxidants to enhance chemical stability where required. Suitable antioxidants include, by way of example only, ascorbic acid, methionine, sodium thiosulfate and sodium metabisulfite. In one embodiment, antioxidants are selected from metal chelating agents, thiol containing compounds and other general stabilizing agents.

Still other useful compositions include one or more auris-acceptable surfactants to enhance physical stability or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In some embodiments, the auris-acceptable pharmaceutical formulations described herein are stable with respect to compound degradation over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months. In other embodiments, the formulations described herein are stable with respect to compound degradation over a period of at least about 1 week. Also described herein are formulations that are stable with respect to compound degradation over a period of at least about 1 month.

In other embodiments, an additional surfactant (co-surfactant) and/or buffering agent is combined with one or more of the pharmaceutically acceptable vehicles previously described herein so that the surfactant and/or buffering agent maintains the product at an optimal pH for stability. Suitable co-surfactants include, but are not limited to: a) natural and synthetic lipophilic agents, e.g., phospholipids, cholesterol, and cholesterol fatty acid esters and derivatives thereof; b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof; c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) cationic surfactants such as cetyltrimethylammonium bromide, and lauryldimethylbenzyl-ammonium chloride.

In a further embodiment, when one or more co-surfactants are utilized in the auris-acceptable formulations of the present disclosure, they are combined, e.g., with a pharmaceutically acceptable vehicle and is present in the final formulation, e.g., in an amount ranging from about 0.1% to about 20%, from about 0.5% to about 10%.

In one embodiment, the surfactant has an HLB value of 0 to 20. In additional embodiments, the surfactant has an HLB value of 0 to 3, of 4 to 6, of 7 to 9, of 8 to 18, of 13 to 15, of 10 to 18.

In one embodiment, diluents are also used to stabilize the otic agent or other pharmaceutical compounds because they provide a more stable environment. Salts dissolved in buffered solutions (which also can provide pH control or maintenance) are utilized as diluents, including, but not limited to a phosphate buffered saline solution. In other embodiments, the gel formulation is isotonic with the EAC. Isotonic formulations are provided by the addition of a tonicity agent. Suitable tonicity agents include, but are not limited to any pharmaceutically acceptable sugar, salt or any combinations or mixtures thereof, such as, but not limited to dextrose and sodium chloride. In further embodiments, the tonicity agents are present in an amount from about 100 mOsm/kg to about 500 mOsm/kg. In some embodiments, the tonicity agent is present in an amount from about 200 mOsm/kg to about 400 mOsm/kg, from about 280 mOsm/kg to about 320 mOsm/kg. The amount of tonicity agents will depend on the target structure of the pharmaceutical formulation, as described herein.

Useful tonicity compositions also include one or more salts in an amount required to bring osmolality of the composition into an acceptable range for application to the EAC. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the auris-acceptable gel formulations disclosed herein alternatively or additionally contain preservatives to prevent microbial growth. Suitable auris-acceptable preservatives for use in the enhanced viscosity formulations described herein include, but are not limited to benzoic acid, boric acid, p-hydroxybenzoates, alcohols, quarternary compounds, stabilized chlorine dioxide, mercurials, such as merfen and thiomersal, mixtures of the foregoing and the like.

In a further embodiment, the preservative is, by way of example only, an otic agent, within the auris-acceptable formulations presented herein. In one embodiment, the formulation includes a preservative such as by way of example only, methyl paraben, sodium bisulfite, sodium thiosulfate, ascorbate, chorobutanol, thimerosal, parabens, benzyl alcohol, phenylethanol and others. In another embodiment, the methyl paraben is at a concentration of about 0.05% to about 1.0%, about 0.1% to about 0.2%. In a further embodiment, the gel is prepared by mixing water, methylparaben, hydroxyethylcellulose and sodium citrate. In a further embodiment, the gel is prepared by mixing water, methylparaben, hydroxyethylcellulose and sodium acetate. In a further embodiment, the mixture is sterilized by autoclaving at 120° C. for about 20 minutes, and tested for pH, methylparaben concentration and viscosity before mixing with the appropriate amount of the otic agent disclosed herein.

Suitable auris-acceptable water soluble preservatives which are employed in the drug delivery vehicle include sodium bisulfite, sodium thiosulfate, ascorbate, chorobutanol, thimerosal, parabens, benzyl alcohol, Butylated hydroxytoluene (BHT), phenylethanol and others. These agents are present, generally, in amounts of about 0.001% to about 5% by weight or, in the amount of about 0.01 to about 2% by weight. In some embodiments, auris-compatible formulations described herein are free of preservatives.

### Excipients

In some embodiments, the auris-acceptable formulations, including gel formulations and viscosity-enhanced formulations, further include excipients, other medicinal or pharmaceutical agents, carriers, adjuvants, such as preserving, stabilizing, wetting or emulsifying agents, solution promoters, salts, solubilizers, an antioxidant, a dispersing agent, a wetting agent, a surfactant, and combinations thereof.

Suitable carriers for use in an auris-acceptable formulation described herein include, but are not limited to, any pharmaceutically acceptable solvent compatible with the targeted auris structure's physiological environment. In other embodiments, the base is a combination of a pharmaceutically acceptable surfactant and solvent.

In some embodiments, other excipients include, sodium stearyl fumarate, diethanolamine cetyl sulfate, isostearate, polyethoxylated castor oil, nonoxyl 10, octoxynol 9, sodium lauryl sulfate, sorbitan esters (sorbitan monolaurate, sorbitan monooleate, sorbitan monopalmitate, sorbitan monostearate, sorbitan sesquioleate, sorbitan trioleate, sorbitan tristearate, sorbitan laurate, sorbitan oleate, sorbitan palmitate, sorbitan stearate, sorbitan dioleate, sorbitan sesqui-isostearate, sorbitan sesquistearate, sorbitan tri-isostearate), lecithin pharmaceutical acceptable salts thereof and combinations or mixtures thereof.

In other embodiments, the carrier is a polysorbate. Polysorbates are nonionic surfactants of sorbitan esters. Polysorbates useful in the present disclosure include, but are not limited to polysorbate 20, polysorbate 40, polysorbate 60, polysorbate 80 (Tween 80) and any combinations or mixtures thereof. In further embodiments, polysorbate 80 is utilized as the pharmaceutically acceptable carrier.

In one embodiment, water-soluble glycerin-based auris-acceptable enhanced viscosity formulations utilized in the preparation of pharmaceutical delivery vehicles comprise at least one otic agent containing at least about 0.1% of the water-soluble glycerin compound or more. In some embodiments, the percentage of otic agent is varied between about 1% and about 95%, between about 5% and about 80%, between about 10% and about 60% or more of the weight or volume of the total pharmaceutical formulation. In some embodiments, the amount of the compound(s) in each therapeutically useful otic agent formulation is prepared in such a way that a suitable dosage will be obtained in any given unit dose of the compound. Factors such as solubility, bioavailability, biological half-life, route of administration, product shelf life, as well as other pharmacological considerations are contemplated herein.

If desired, the auris-acceptable pharmaceutical gels also contain co-solvents, preservatives, cosolvents, ionic strength and osmolality adjustors and other excipients in addition to buffering agents. Suitable auris-acceptable water soluble buffering agents are alkali or alkaline earth metal carbonates, phosphates, bicarbonates, citrates, borates, acetates, succinates and the like, such as sodium phosphate, citrate, borate, acetate, bicarbonate, carbonate and tromethamine (TRIS). These agents are present in amounts sufficient to maintain the pH of the system at 7.4±0.2 and preferably, 7.4. As such, the buffering agent is as much as 5% on a weight basis of the total composition.

Cosolvents are used to enhance otic agent solubility, however, some otic agents or other pharmaceutical compounds are insoluble. These are often suspended in the polymer vehicle with the aid of suitable suspending or viscosity enhancing agents.

Moreover, some pharmaceutical excipients, diluents or carriers are potentially ototoxic. For example, benzalkonium chloride, a common preservative, is ototoxic and therefore potentially harmful if introduced into the ear. In formulating a controlled release otic agent formulation, it is advised to avoid or combine the appropriate excipients, diluents or carriers to lessen or eliminate potential ototoxic components from the formulation, or to decrease the amount of such excipients, diluents or carriers. Optionally, a controlled release otic agent formulation includes otoprotective agents, such as antioxidants, alpha lipoic acid, calcium, fosfomycin or iron chelators, to counteract potential ototoxic effects that may arise from the use of specific therapeutic agents or excipients, diluents or carriers.

### Modes of Treatment

Dosing Methods and Schedules

Drugs delivered to the EAC are generally administered by syringing. In some embodiments, the delivery system is a syringe and needle apparatus that is capable of unloading the otic compositions or formulations disclosed herein onto the surface of the tympanic membrane or into the external auditory canal. In some embodiments, the needle on the syringe is wider than a 18 gauge needle. In another embodiment, the needle gauge is from 18 gauge to 31 gauge. In a further embodiment, the needle gauge is from 25 gauge to 30 gauge. Depending upon the thickness or viscosity of the otic agent compositions or formulations, the gauge level of the syringe or hypodermic needle may be varied accordingly. In another embodiment, the internal diameter of the needle can be increased by reducing the wall thickness of the needle (commonly referred as thin wall or extra thin wall needles) to reduce the possibility of needle clogging while maintaining an adequate needle gauge.

In some embodiments, the needle is a needle used for instant delivery of the gel formulation. The needle may be a single use needle or a disposable needle. In some embodiments, a syringe may be used for delivery of the pharmaceutically acceptable gel-based otic agent-containing compositions as disclosed herein wherein the syringe has a press-fit (Luer) or twist-on (Luer-lock) fitting. In one embodiment, the syringe is a hypodermic syringe. In another embodiment, the syringe is made of plastic or glass. In yet another embodiment, the hypodermic syringe is a single use syringe. In a further embodiment, the glass syringe is capable of being sterilized. In yet a further embodiment, the sterilization occurs through an autoclave. In another embodiment, the syringe comprises a cylindrical syringe body wherein the gel formulation is stored before use. In other embodiments, the syringe comprises a cylindrical syringe body wherein the otic agent pharmaceutically acceptable gel-based compositions as disclosed herein is stored before use which conveniently allows for mixing with a suitable pharmaceutically acceptable buffer. In other embodiments, the syringe may contain other excipients, stabilizers, suspending agents, diluents or a combination thereof to stabilize or otherwise stably store the otic agent or other pharmaceutical compounds contained therein.

In some embodiments, the syringe comprises a cylindrical syringe body wherein the body is compartmentalized in that each compartment is able to store at least one component of the auris-acceptable otic agent gel formulation. In a further embodiment, the syringe having a compartmentalized body allows for mixing of the components prior to injection into the EAC. In other embodiments, the delivery system comprises multiple syringes, each syringe of the multiple syringes contains at least one component of the gel formulation such that each component is pre-mixed prior to injection or is mixed subsequent to injection. In a further embodiment, the syringes disclosed herein comprise at least one reservoir wherein the at least one reservoir comprises an otic agent, or a pharmaceutically acceptable buffer, or a viscosity enhancing agent, such as a gelling agent or a combination thereof. Commercially available injection devices are optionally employed in their simplest form as ready-to-use plastic syringes with a syringe barrel, needle assembly with a needle, plunger with a plunger rod, and holding flange, to perform an injection.

In some embodiments, the auris-acceptable compositions or formulations disclosed herein is delivered or injected onto the surface of the tympanic membrane or into the external auditory canal without the use of a needle. In some embodiments, the auris-acceptable compositions or formulations disclosed herein is delivered or injected onto the surface of the tympanic membrane or into the external auditory canal using a syringe. In some embodiments, the auris-acceptable compositions or formulations disclosed herein is delivered or injected onto the surface of the tympanic membrane or into the external auditory canal using a dropper, or any delivery device capable of deliver the disclosed auris-acceptable compositions onto the targeted area.

The auris-acceptable compositions or formulations containing the otic agent compound(s) described herein are administered for prophylactic and/or therapeutic treatments. In therapeutic applications, the otic agent compositions are administered to a patient already suffering from a condition or disorder, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician.

### Frequency of Administration

In some embodiments, a compositon disclosed herein is administered to an individual in need thereof once. In some embodiments, a compositon disclosed herein is administered to an individual in need thereof more than once. In some embodiments, a first administration of a composition disclosed herein is followed by a second administration of a composition disclosed herein. In some embodiments, a first administration of a composition disclosed herein is followed by a second and third administration of a composition disclosed herein. In some embodiments, a first administration of a composition disclosed herein is followed by a second, third, and fourth administration of a composition disclosed herein. In some embodiments, a first administration of a composition disclosed herein is followed by a second, third, fourth, and fifth administration of a composition disclosed herein. In some embodiments, a first administration of a composition disclosed herein is followed by a drug holiday.

The number of times a composition is administered to an individual in need thereof depends on the discretion of a medical professional, the disorder, the severity of the disorder, and the individuals's response to the formulation. In some embodiments, a composition disclosed herein is administered once to an individual in need thereof with a mild acute condition. In some embodiments, a composition disclosed herein is administered more than once to an individual in need thereof with a moderate or severe acute condition. In the case wherein the patient's condition does not improve, upon the doctor's discretion the administration of an otic agent may be administered chronically, that is, for an extended period of time, including throughout the duration of the patient's life in order to ameliorate or otherwise control or limit the symptoms of the patient's disease or condition.

In the case wherein the patient's condition does not improve, upon the doctor's discretion the administration of the otic agent compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the otic agent compounds may be given continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's otic conditions has occurred, a maintenance otic agent dose is administered if necessary. Subsequently, the dosage or the frequency of administration, or both, is optionally reduced, as a function of the symptoms, to a level at which the improved disease, disorder or condition is retained. In certain embodiments, patients require intermittent treatment on a long-term basis upon any recurrence of symptoms.

The amount of otic agent that will correspond to such an amount will vary depending upon factors such as the particular compound, disease condition and its severity, according to the particular circumstances surrounding the case, including, e.g., the specific otic agent being administered, the route of administration, the condition being treated, the target area being treated, and the subject or host being treated. In general, however, doses employed for adult human treatment will typically be in the range of 0.02-50 mg per administration, preferably 1-15 mg per administration. The desired dose is presented in a single dose or as divided doses administered simultaneously (or over a short period of time) or at appropriate intervals.

In some embodiments, the initial administration is a particular otic agent and the subsequent administration a different formulation or otic agent.

### Pharmacokinetics of Controlled Release Formulations

In one embodiment, the formulations disclosed herein additionally provides an immediate release of an otic agent from the composition, or within 1 minute, or within 5 minutes, or within 10 minutes, or within 15 minutes, or within 30 minutes, or within 60 minutes or within 90 minutes. In other embodiments, a therapeutically effective amount of at least one otic agent is released from the composition immediately, or within 1 minute, or within 5 minutes, or within 10 minutes, or within 15 minutes, or within 30 minutes, or within 60 minutes or within 90 minutes. In certain embodiments the composition comprises an auris-pharmaceutically acceptable gel formulation providing immediate release of at least one otic agent. Additional embodiments of the formulation may also include an agent that enhances the viscosity of the formulations included herein.

In other or further embodiments, the formulation provides an extended release formulation of at least one otic agent. In certain embodiments, diffusion of at least one otic agent from the formulation occurs for a time period exceeding 5 minutes, or 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year. In other embodiments, a therapeutically effective amount of at least one otic agent is released from the formulation for a time period exceeding 5 minutes, or 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

In other embodiments, the formulation provides both an immediate release and an extended release formulation of an otic agent. In yet other embodiments, the formulation contains a 0.25:1 ratio, or a 0.5:1 ratio, or a 1:1 ratio, or a 1:2 ratio, or a 1:3, or a 1:4 ratio, or a 1:5 ratio, or a 1:7 ratio, or a 1:10 ratio, or a 1:15 ratio, or a 1:20 ratio of immediate release and extended release formulations. In a further embodiment the formulation provides an immediate release of a first otic agent and an extended release of a second otic agent or other therapeutic agent. In yet other embodiments, the formulation provides an immediate release and extended release formulation of at least one otic agent, and at least one therapeutic agent. In some embodiments, the formulation provides a 0.25:1 ratio, or a 0.5:1 ratio, or a 1:1 ratio, or a 1:2 ratio, or a 1:3, or a 1:4 ratio, or a 1:5 ratio, or a 1:7 ratio, or a 1:10 ratio, or a 1:15 ratio, or a 1:20 ratio of immediate release and extended release formulations of a first otic agent and second therapeutic agent, respectively.

In a specific embodiment the formulation provides a therapeutically effective amount of at least one otic agent at the treatment site (e.g. EAC) with essentially no systemic exposure. In an additional embodiment the formulation provides a therapeutically effective amount of at least one otic agent at the treatment site with essentially no detectable systemic exposure. In other embodiments, the formulation provides a therapeutically effective amount of at least one otic agent at the treatment site with little or no detectable detectable systemic exposure.

The combination of immediate release, delayed release and/or extended release otic agent compositions or formulations may be combined with other pharmaceutical agents, as well as the excipients, diluents, stabilizers, tonicity agents and other components disclosed herein. As such, depending upon the otic agent used, the thickness or viscosity desired, or the mode of delivery chosen, alternative aspects of the embodiments disclosed herein are combined with the immediate release, delayed release and/or extended release embodiments accordingly.

In certain embodiments, the pharmacokinetics of the otic agent formulations described herein are determined by injecting the formulation into the EAC or on or near the surface of the tympanic membrane of a test animal (including by way of example, a guinea pig or a chinchilla). At a determined period of time (e.g., 6 hours, 12 hours, 1 day, 2 days, 3 days, 4 days, 5 days, 6 days, and 7 days for testing the pharmacokinetics of a formulation over a 1 week period), the test animal is euthanized and the level of otic agent is measured in the ear or in other organs. In addition, the systemic level of the otic agent is measured by withdrawing a blood sample from the test animal. In order to determine whether the formulation impedes hearing, the hearing of the test animal is optionally tested.

### Kits/Articles of Manufacture

The disclosure also provides kits for modulating the production of cerumen and treatment of ceruminosis and ceruminosis associated diseases in a mammal. Such kits generally will comprise one or more of the otic agent controlled-release compositions disclosed herein, and instructions for using the kit. The disclosure also contemplates the use of one or more of the otic agent controlled-release compositions, in the manufacture of medicaments for treating, abating, reducing, or ameliorating the symptoms of a disease, dysfunction, or disorder in a mammal, such as a human that has, is suspected of having, or at risk for developing ceruminosis.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

The articles of manufacture provided herein contain packaging materials. Packaging materials for use in packaging pharmaceutical products are also presented herein. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of otic agent formulations compositions provided herein are contemplated as are a variety of treatments for any disease, disorder, or condition that would benefit by controlled release administration of an otic agent to the EAC.

In some embodiments, a kit includes one or more additional containers, each with one or more of various materials (such as reagents, optionally in concentrated form, and/or devices) desirable from a commercial and user standpoint for use of a formulation described herein. Non-limiting examples of such materials include, but not limited to, buffers, diluents, filters, needles, syringes; carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

In certain embodiments, the pharmaceutical compositions are presented in a pack or dispenser device which contains one or more unit dosage forms containing a compound provided herein. In another embodiment, the pack for example contains metal or plastic foil, such as a blister pack. In a further embodiment, the pack or dispenser device is accompanied by instructions for administration. In yet a further embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. In another embodiment, such notice, for example, is the labeling approved by the U.S. Food and Drug Administration for prescription drugs, or the approved product insert. In yet another embodiment, compositions containing a compound provided herein formulated in a compatible pharmaceutical carrier are also prepared, placed in an appropriate container, and labeled for treatment of an indicated condition.

### Examples

### Example 1

Exemplary compositions for preparation of thermoreversible gel otic formulations are described in Tables 1-12.

TABLE 1
Thermoreversible Gel Choline Ester or Carbamate Formulation

| | Quantity (g) for | Concentration | |
|---|---|---|---|
| | 1000 mL | in 1000 mL | |
| Ingredient | solution | aqueous solution | |
| Choline ester or carbamate | 1-200 | 0.1-20 | (wt %) |
| (e.g. acetylcholine or carbachol) | | | |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 2
Thermoreversible Gel Choline Ester or Carbamate
Formulation Containing Additional Active Agents

| | Quantity (g) for | Concentration | |
|---|---|---|---|
| | 1000 mL | in 1000 mL | |
| Ingredient | solution | aqueous solution | |
| Choline ester or carbamate | 1-200 | 0.1-20 | (wt %) |
| (e.g. acetylcholine or carbachol) | | | |
| squalene | 10-200 | 1-20 | (wt %) |
| lanosterol | 10-200 | 1-20 | (wt %) |
| cholesterol | 10-200 | 1-20 | (wt %) |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 3
Thermoreversible Gel Plant Alkaloid Formulation

| | Quantity (g) for | Concentration | |
|---|---|---|---|
| | 1000 mL | in 1000 mL | |
| Ingredient | solution | aqueous solution | |
| plant alkaloid | 1-200 | 0.1-20 | (wt %) |
| (e.g. pilocarpine) | | | |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 4
Thermoreversible Gel Plant Alkaloid Formulation
Containing Additional Active Agents

| | Quantity (g) for | Concentration | |
|---|---|---|---|
| | 1000 mL | in 1000 mL | |
| Ingredient | solution | aqueous solution | |
| plant alkaloid | 1-200 | 0.1-20 | (wt %) |
| (e.g. pilocarpine) | | | |
| squalene | 10-200 | 1-20 | (wt %) |
| lanosterol | 10-200 | 1-20 | (wt %) |
| cholesterol | 10-200 | 1-20 | (wt %) |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 5
Thermoreversible Gel Reversible Cholinesterase
Inhibitor Formulation

| | Quantity (g) for | Concentration | |
|---|---|---|---|
| | 1000 mL | in 1000 mL | |
| Ingredient | solution | aqueous solution | |
| reversible cholinesterase | 1-200 | 0.1-20 | (wt %) |
| inhibitor | | | |
| (e.g. neostigmine | | | |
| or physostigmine) | | | |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 6
Thermoreversible Gel Reversible Cholinesterase Inhibitor
Formulation Containing Additional Active Agents

| | Quantity (g) for | Concentration | |
|---|---|---|---|
| | 1000 mL | in 1000 mL | |
| Ingredient | solution | aqueous solution | |
| reversible cholinesterase | 1-200 | 0.1-20 | (wt %) |
| inhibitor | | | |
| (e.g. neostigmine | | | |
| or physostigmine) | | | |
| squalene | 10-200 | 1-20 | (wt %) |
| lanosterol | 10-200 | 1-20 | (wt %) |
| cholesterol | 10-200 | 1-20 | (wt %) |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 7
Thermoreversible Gel Acetylcholine Release Promoter Formulation

| | Quantity (g) for | Concentration | |
|---|---|---|---|
| | 1000 mL | in 1000 mL | |
| Ingredient | solution | aqueous solution | |
| acetylcholine release promoter | 1-200 | 0.1-20 | (wt %) |
| (e.g. droperidol, | | | |
| resperidone, or trazodone) | | | |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 8
Thermoreversible Gel Acetylcholine Release Promoter
Formulation Containing Additional Active Agents

| | Quantity (g) for | Concentration | |
|---|---|---|---|
| | 1000 mL | in 1000 mL | |
| Ingredient | solution | aqueous solution | |
| acetylcholine release promoter | 1-200 | 0.1-20 | (wt %) |
| (e.g. droperidol, | | | |
| resperidone, or trazodone) | | | |
| squalene | 10-200 | 1-20 | (wt %) |
| lanosterol | 10-200 | 1-20 | (wt %) |
| cholesterol | 10-200 | 1-20 | (wt %) |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 9
Thermoreversible Gel Anti-adrenergic Formulation

| | Quantity (g) for | Concentration | |
|---|---|---|---|
| | 1000 mL | in 1000 mL | |
| Ingredient | solution | aqueous solution | |
| anti-adrenergic | 1-200 | 0.1-20 | (wt %) |
| (e.g. clonidine, propranolol, | | | |
| atenolol, or prazosin) | | | |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 10
Thermoreversible Gel Anti-adrenergic Formulation
Containing Additional Active Agents

| | Quantity (g) for | Concentration | |
|---|---|---|---|
| | 1000 mL | in 1000 mL | |
| Ingredient | solution | aqueous solution | |
| anti-adrenergic | 1-200 | 0.1-20 | (wt %) |
| (e.g. clonidine, propranolol, | | | |
| atenolol, or prazosin) | | | |
| squalene | 10-200 | 1-20 | (wt %) |
| lanosterol | 10-200 | 1-20 | (wt %) |
| cholesterol | 10-200 | 1-20 | (wt %) |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 11
Thermoreversible Gel Sympathomimetic Formulation

| | Quantity (g) for | Concentration | |
|---|---|---|---|
| | 1000 mL | in 1000 mL | |
| Ingredient | solution | aqueous solution | |
| sympathomimetic | 1-200 | 0.1-20 | (wt %) |
| (e.g. norepinephrine | | | |
| or dopamine) | | | |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

TABLE 12
Thermoreversible Gel Sympathomimetic Formulation
Containing Additional Active Agents

| | Quantity (g) for | Concentration | |
|---|---|---|---|
| | 1000 mL | in 1000 mL | |
| Ingredient | solution | aqueous solution | |
| sympathomimetic | 1-200 | 0.1-20 | (wt %) |
| (e.g. norepinephrine | | | |
| or dopamine) | | | |
| squalene | 10-200 | 1-20 | (wt %) |
| lanosterol | 10-200 | 1-20 | (wt %) |
| cholesterol | 10-200 | 1-20 | (wt %) |
| Buffering agent | 3-10 | 0.3-1 | (wt %) |
| (e.g. tromethamine) | | | |
| pH adjusting agent (e.g. HCl) | — | q.s. for pH = 5.5-9.0 | |
| Poloxamer 407 | 100-250 | 10-25 | (wt %) |

| | | |
|---|---|---|
| Osmolarity modifier (e.g. NaCl) | — | q.s. 150-500 mOsm/L |

### Example 2—Preparation of a Thermoreversible Gel Formulation Containing Choline Ester or Carbamate

| | Quantity (mg/g of |
|---|---|
| Ingredient | formulation) |
| Choline ester or carbamate | 60.0 |
| NaCl | 100.0 |
| Poloxamer 407 | 160.0 |
| TRIS HCl buffer (0.1M) | 680.0 |

A 10-g batch of gel formulation containing 6.0% of choline ester or carbamate (e.g. acetylcholine or carbachol) is prepared by suspending 1.60 g of Poloxamer 407 (BASF Corp.) in 5.00 g of TRIS HCl buffer (0.1 M) and the components are mixed under agitation overnight at 4° C. to ensure complete dissolution. The choline ester or carbamate (e.g. acetylcholine or carbachol) (600.0 mg), NaCl (1 g) and additional TRIS HCl buffer (0.1 M) (1.80 g) is added and further stirring allowed until complete dissolution is observed. The mixture is maintained below room temperature until use.

### Preparation of a Thermoreversible Gel Formulation Containing Choline Ester or Carbamate

| | Quantity (mg/g of |
|---|---|
| Ingredient | formulation) |
| Choline ester or carbamate | 60.0 |
| squalene | 60.0 |
| lanosterol | 60.0 |
| cholesterol | 60.0 |
| NaCl | 100.0 |
| Poloxamer 407 | 160.0 |
| TRIS HCl buffer (0.1M) | 500.0 |

A 10-g batch of gel formulation containing 6.0% of choline ester or carbamate (e.g. acetylcholine or carbachol) is prepared by suspending 1.60 g of Poloxamer 407 (BASF Corp.) in 4.00 g of TRIS HCl buffer (0.1 M) and the components are mixed under agitation overnight at 4° C. to ensure complete dissolution. The choline ester or carbamate (e.g. acetylcholine or carbachol) (600.0 mg), squalene (600.0 mg), lanosterol (600.0 mg), cholesterol (600.0 mg), NaCl (1 g) and additional TRIS HCl buffer (0.1 M) (100 mg) is added and further stirring allowed until complete dissolution is observed. The mixture is maintained below room temperature until use.

### Example 3—Preparation of a Thermoreversible Gel Formulation Containing Plant Alkaloid

| | Quantity (mg/g of |
|---|---|
| Ingredient | formulation) |
| Plant alkaloid | 60.0 |
| NaCl | 100.0 |
| Poloxamer 407 | 160.0 |
| TRIS HCl buffer (0.1M) | 680.0 |

A 10-g batch of gel formulation containing 6.0% of plant alkaloid (e.g. pilocarpine) is prepared by suspending 1.60 g of Poloxamer 407 (BASF Corp.) in 5.00 g of TRIS HCl buffer (0.1 M) and the components are mixed under agitation overnight at 4° C. to ensure complete dissolution. The plant alkaloid (e.g. pilocarpine) (600.0 mg), NaCl (1 g) and additional TRIS HCl buffer (0.1 M) (1.80 g) is added and further stirring allowed until complete dissolution is observed. The mixture is maintained below room temperature until use.

### Preparation of a Thermoreversible Gel Formulation Containing Plant Alkaloid

| | Quantity (mg/g of |
|---|---|
| Ingredient | formulation) |
| Plant alkaloid | 60.0 |
| squalene | 60.0 |
| lanosterol | 60.0 |
| cholesterol | 60.0 |
| NaCl | 100.0 |
| Poloxamer 407 | 160.0 |
| TRIS HCl buffer (0.1M) | 500.0 |

A 10-g batch of gel formulation containing 6.0% of plant alkaloid (e.g. pilocarpine) is prepared by suspending 1.60 g of Poloxamer 407 (BASF Corp.) in 4.00 g of TRIS HCl buffer (0.1 M) and the components are mixed under agitation overnight at 4° C. to ensure complete dissolution. The plant alkaloid (e.g. pilocarpine) (600.0 mg), squalene (600.0 mg), lanosterol (600.0 mg), cholesterol (600.0 mg), NaCl (1 g) and additional TRIS HCl buffer (0.1 M) (100 mg) is added and further stirring allowed until complete dissolution is observed. The mixture is maintained below room temperature until use.

### Example 4—Preparation of a Thermoreversible Gel Formulation Containing Cholinesterase Inhibitor

| | Quantity (mg/g of |
|---|---|
| Ingredient | formulation) |
| Reversible cholinesterase inhibitor | 60.0 |
| NaCl | 100.0 |
| Poloxamer 407 | 160.0 |
| TRIS HCl buffer (0.1M) | 680.0 |

A 10-g batch of gel formulation containing 6.0% of reversible cholinesterase inhibitor (e.g. neostigmine or physostigmine) is prepared by suspending 1.60 g of Poloxamer 407 (BASF Corp.) in 5.00 g of TRIS HCl buffer (0.1 M) and the components are mixed under agitation overnight at 4° C. to ensure complete dissolution. The reversible cholinesterase inhibitor (e.g. neostigmine or physostigmine) (600.0 mg), NaCl (1 g) and additional TRIS HCl buffer (0.1 M) (1.80 g) is added and further stirring allowed until complete dissolution is observed. The mixture is maintained below room temperature until use.

### Preparation of a Thermoreversible Gel Formulation Containing Cholinesterase Inhibitor

| | Quantity (mg/g of |
|---|---|
| Ingredient | formulation) |
| Reversible cholinesterase inhibitor | 60.0 |
| squalene | 60.0 |
| lanosterol | 60.0 |
| cholesterol | 60.0 |
| NaCl | 100.0 |
| Poloxamer 407 | 160.0 |
| TRIS HCl buffer (0.1M) | 500.0 |

A 10-g batch of gel formulation containing 6.0% of reversible cholinesterase inhibitor (e.g. neostigmine or physostigmine) is prepared by suspending 1.60 g of Poloxamer 407 (BASF Corp.) in 4.00 g of TRIS HCl buffer (0.1 M) and the components are mixed under agitation overnight at 4° C. to ensure complete dissolution. The reversible cholinesterase inhibitor (e.g. neostigmine or physostigmine) (600.0 mg), squalene (600.0 mg), lanosterol (600.0 mg), cholesterol (600.0 mg), NaCl (1 g) and additional TRIS HCl buffer (0.1 M) (100 mg) is added and further stirring allowed until complete dissolution is observed. The mixture is maintained below room temperature until use.

### Example 5—Preparation of a Thermoreversible Gel Formulation Containing Acetylcholine Release Promoter

| | Quantity (mg/g of |
|---|---|
| Ingredient | formulation) |
| Acetylcholine release promoter | 60.0 |
| NaCl | 100.0 |
| Poloxamer 407 | 160.0 |
| TRIS HCl buffer (0.1M) | 680.0 |

A 10-g batch of gel formulation containing 6.0% of acetylcholine release promoter (e.g. droperidol, resperidone, or trazodone) is prepared by suspending 1.60 g of Poloxamer 407 (BASF Corp.) in 5.00 g of TRIS HCl buffer (0.1 M) and the components are mixed under agitation overnight at 4° C. to ensure complete dissolution. The acetylcholine release promoter (e.g. droperidol, resperidone, or trazodone) (600.0 mg), NaCl (1 g) and additional TRIS HCl buffer (0.1 M) (1.80 g) is added and further stirring allowed until complete dissolution is observed. The mixture is maintained below room temperature until use.

### Preparation of a Thermoreversible Gel Formulation Containing Acetylcholine Release Promoter

| | Quantity (mg/g of |
|---|---|
| Ingredient | formulation) |
| Acetylcholine release promoter | 60.0 |
| squalene | 60.0 |
| lanosterol | 60.0 |
| cholesterol | 60.0 |
| NaCl | 100.0 |
| Poloxamer 407 | 160.0 |
| TRIS HCl buffer (0.1M) | 500.0 |

A 10-g batch of gel formulation containing 6.0% of acetylcholine release promoter (e.g. droperidol, resperidone, or trazodone) is prepared by suspending 1.60 g of Poloxamer 407 (BASF Corp.) in 4.00 g of TRIS HCl buffer (0.1 M) and the components are mixed under agitation overnight at 4° C. to ensure complete dissolution. The acetylcholine release promoter (e.g. droperidol, resperidone, or trazodone) (600.0 mg), squalene (600.0 mg), lanosterol (600.0 mg), cholesterol (600.0 mg), NaCl (1 g) and additional TRIS HCl buffer (0.1 M) (100 mg) is added and further stirring allowed until complete dissolution is observed. The mixture is maintained below room temperature until use.

### Example 6—Preparation of a Thermoreversible Gel Formulation Containing Anti-adrenergic

| | Quantity (mg/g of |
|---|---|
| Ingredient | formulation) |
| Anti-adrenergic | 60.0 |
| NaCl | 100.0 |
| Poloxamer 407 | 160.0 |
| TRIS HCl buffer (0.1M) | 680.0 |

A 10-g batch of gel formulation containing 6.0% of anti-adrenergic (e.g. clonidine, propranolol, atenolol, or prazosin) is prepared by suspending 1.60 g of Poloxamer 407 (BASF Corp.) in 5.00 g of TRIS HCl buffer (0.1 M) and the components are mixed under agitation overnight at 4° C. to ensure complete dissolution. The anti-adrenergic (e.g. clonidine, propranolol, atenolol, or prazosin) (600.0 mg), NaCl (1 g) and additional TRIS HCl buffer (0.1 M) (1.80 g) is added and further stirring allowed until complete dissolution is observed. The mixture is maintained below room temperature until use.

### Preparation of a Thermoreversible Gel Formulation Containing Anti-Adrenergic

| | Quantity (mg/g of |
|---|---|
| Ingredient | formulation) |
| Anti-adrenergic | 60.0 |
| squalene | 60.0 |
| lanosterol | 60.0 |
| cholesterol | 60.0 |
| NaCl | 100.0 |
| Poloxamer 407 | 160.0 |
| TRIS HCl buffer (0.1M) | 500.0 |

A 10-g batch of gel formulation containing 6.0% of anti-adrenergic (e.g. clonidine, propranolol, atenolol, or prazosin) is prepared by suspending 1.60 g of Poloxamer 407 (BASF Corp.) in 4.00 g of TRIS HCl buffer (0.1 M) and the components are mixed under agitation overnight at 4° C. to ensure complete dissolution. The anti-adrenergic (e.g. clonidine, propranolol, atenolol, or prazosin) (600.0 mg), squalene (600.0 mg), lanosterol (600.0 mg), cholesterol (600.0 mg), NaCl (1 g) and additional TRIS HCl buffer (0.1 M) (100 mg) is added and further stirring allowed until complete dissolution is observed. The mixture is maintained below room temperature until use.

### Example 7—Preparation of a Thermoreversible Gel Formulation Containing Sympathomimetic

| | Quantity (mg/g of |
|---|---|
| Ingredient | formulation) |
| sympathomimetic | 60.0 |
| NaCl | 100.0 |
| Poloxamer 407 | 160.0 |
| TRIS HCl buffer (0.1M) | 680.0 |

A 10-g batch of gel formulation containing 6.0% of sympathomimetic (e.g. norepinephrine or dopamine) is prepared by suspending 1.60 g of Poloxamer 407 (BASF Corp.) in 5.00 g of TRIS HCl buffer (0.1 M) and the components are mixed under agitation overnight at 4° C. to ensure complete dissolution. The sympathomimetic (e.g. norepinephrine or dopamine) (600.0 mg), NaCl (1 g) and additional TRIS HCl buffer (0.1 M) (1.80 g) is added and further stirring allowed until complete dissolution is observed. The mixture is maintained below room temperature until use.

### Preparation of a Thermoreversible Gel Formulation Containing Sympathomimetic

| | Quantity (mg/g of |
|---|---|
| Ingredient | formulation) |
| sympathomimetic | 60.0 |
| squalene | 60.0 |
| lanosterol | 60.0 |
| cholesterol | 60.0 |
| NaCl | 100.0 |
| Poloxamer 407 | 160.0 |
| TRIS HCl buffer (0.1M) | 500.0 |

A 10-g batch of gel formulation containing 6.0% of sympathomimetic (e.g. norepinephrine or dopamine) is prepared by suspending 1.60 g of Poloxamer 407 (BASF Corp.) in 4.00 g of TRIS HCl buffer (0.1 M) and the components are mixed under agitation overnight at 4° C. to ensure complete dissolution. The sympathomimetic (e.g. norepinephrine or dopamine) (600.0 mg), squalene (600.0 mg), lanosterol (600.0 mg), cholesterol (600.0 mg), NaCl (1 g) and additional TRIS HCl buffer (0.1 M) (100 mg) is added and further stirring allowed until complete dissolution is observed. The mixture is maintained below room temperature until use.

### Example 8—Preparation of a Thermoreversible Gel Composition Comprising Micronized Choline Ester or Carbamate Powder and Micronized Dexamethasone Powder

| | Quantity (mg/g of |
|---|---|
| Ingredient | formulation) |
| Choline Ester or Carbamate | 15.0 |
| dexamethasone | 15.0 |
| BHT | 0.002 |
| Poloxamer 407 | 160.0 |
| PBS buffer (0.1M) | 9.0 |

A 10-g batch of gel formulation containing 2.0% micronized choline ester or carbamate (e.g. acetylcholine or carbachol) and micronized dexamethasone is prepared. Micronized choline ester or carbamate (e.g. acetylcholine or carbachol), micronized dexamethasone, 13.8 mg of sodium phosphate dibasic dihydrate USP (Fisher Scientific.)+3.1 mg of sodium phosphate monobasic monohydrate USP (Fisher Scientific.)+74 mg of sodium chloride USP (Fisher Scientific.) is dissolved with 8.2 g of sterile filtered DI water and the pH is adjusted to 7.4 with 1 M NaOH. The buffer solution is chilled down and 1.6 g of poloxamer 407 (BASF Corp., containing approximately 100 ppm of BHT) is sprinkled into the chilled PBS solution while mixing. Solution is mixed until all the poloxamer is dissolved. The poloxamer is sterile filtered using a 33 mm PVDF 0.22 μm sterile syringe filter (Millipore Corp.) and delivered to 2 mL sterile glass vials (Wheaton) in an aseptic environment, the vials are closed with sterile butyl rubber stoppers (Kimble) and crimped sealed with 13 min Al seals (Kimble). 20 mg of micronized choline ester or carbamate (e.g. acetylcholine or carbachol) and dexamethasone is placed in separate clean depyrogenated vials, the vials are closed with sterile butyl rubber stoppers (Kimble) and crimped sealed with 13 mm Al seals (Kimble), vials are dry heat sterilized (Fisher Scientific Isotemp oven) for 7 hours at 140° C. Before administration for the experiments described herein, 1 mL of the cold poloxamer solution is delivered to a vial containing 20 mg of sterile micronized choline ester or carbamate (e.g. acetylcholine or carbachol) and dexamethasone using a 21G needle (Becton Dickinson) attached to a 1 mL sterile syringe (Becton Dickinson), suspension mixed well by shaking to ensure homogeneity of the suspension. The suspension is then withdrawn with the 21G syringe and the needle is switched to a 27 G needle for administration.

### Preparation of a Thermoreversible Gel Composition Comprising Micronized Choline Ester or Carbamate Powder, Micronized Dexamethasone Powder, and Powders of Additional Active Agents

| | Quantity (mg/g of |
|---|---|
| Ingredient | formulation) |
| Choline Ester or Carbamate | 15.0 |
| dexamethasone | 15.0 |
| squalene | 60.0 |
| lanosterol | 60.0 |
| cholesterol | 60.0 |
| BHT | 0.002 |
| Poloxamer 407 | 160.0 |
| PBS buffer (0.1M) | 9.0 |

A 10-g batch of gel formulation containing 2.0% micronized choline ester or carbamate (e.g. acetylcholine or carbachol), micronized dexamethasone, micronized squalene, micronized lanosterl, micronized cholesterol is prepared. Micronized choline ester or carbamate (e.g. acetylcholine or carbachol), micronized dexamethasone, micronized squalene, micronized lanosterol, micronized cholesterol, 13.8 mg of sodium phosphate dibasic dihydrate USP (Fisher Scientific.)+3.1 mg of sodium phosphate monobasic monohydrate USP (Fisher Scientific.)+74 mg of sodium chloride USP (Fisher Scientific.) is dissolved with 8.2 g of sterile filtered DI water and the pH is adjusted to 7.4 with 1 M NaOH. The buffer solution is chilled down and 1.6 g of poloxamer 407 (BASF Corp., containing approximately 100 ppm of BHT) is sprinkled into the chilled PBS solution while mixing. Solution is mixed until all the poloxamer is dissolved. The poloxamer is sterile filtered using a 33 mm PVDF 0.22 μm sterile syringe filter (Millipore Corp.) and delivered to 2 mL sterile glass vials (Wheaton) in an aseptic environment, the vials are closed with sterile butyl rubber stoppers (Kimble) and crimped sealed with 13 mm Al seals (Kimble). 20 mg of micronized choline ester or carbamate (e.g. acetylcholine or carbachol), dexamethasone, squalene, lanosterol, and cholesterol is placed in separate clean depyrogenated vials, the vials are closed with sterile butyl rubber stoppers (Kimble) and crimped sealed with 13 mm Al seals (Kimble), vials are dry heat sterilized (Fisher Scientific Isotemp oven) for 7 hours at 140° C. Before administration for the experiments described herein, 1 mL of the cold poloxamer solution is delivered to a vial containing 20 mg of sterile micronized choline ester or carbamate (e.g. acetylcholine or carbachol), dexamethasone, squalene, lanosterol, and cholesterol using a 21G needle (Becton Dickinson) attached to a 1 mL sterile syringe (Becton Dickinson), suspension mixed well by shaking to ensure homogeneity of the suspension. The suspension is then withdrawn with the 21G syringe and the needle is switched to a 27 G needle for administration.

### Example 17—Effect of pH on Degradation Products for Autoclaved 16% Poloxamer 407/2% Otic Agent in PBS Buffer

A stock solution of a 16% poloxamer 407/2% otic agent is prepared by dissolving 351.4 mg of sodium chloride (Fisher Scientific), 302.1 mg of sodium phosphate dibasic anhydrous (Fisher Scientific), 122.1 mg of sodium phosphate monobasic anhydrous (Fisher Scientific) and an appropriate amount of an otic agent with 79.3 g of sterile filtered DI water. The solution is cooled down in a ice chilled water bath and then 16.05 g of poloxamer 407 is sprinkled into the cold solution while mixing. The mixture is further mixed until the poloxamer is completely dissolved. The pH for this solution is measured.

16% poloxamer 407/2% otic agent in PBS pH of 5.3. Take an aliquot (approximately 30 mL) of the above solution and adjust the pH to 5.3 by the addition of 1 M HCl.

16% poloxamer 407/2% otic agent in PBS pH of 8.0. Take an aliquot (approximately 30 mL) of the above stock solution and adjust the pH to 8.0 by the addition of 1 M NaOH.

A PBS buffer (pH 7.3) is prepared by dissolving 805.5 mg of sodium chloride (Fisher Scientific), 606 mg of sodium phosphate dibasic anhydrous (Fisher Scientific), 247 mg of sodium phosphate monobasic anhydrous (Fisher Scientific), then QS to 200 g with sterile filtered DI water.

A 2% solution of an otic agent in PBS pH 7.3 is prepared by dissolving an appropriate amount of the otic agent in the PBS buffer and QS to 10 g with PBS buffer.

One mL samples are individually placed in 3 mL screw cap glass vials (with rubber lining) and closed tightly. The vials are placed in a Market Forge-sterilmatic autoclave (settings, slow liquids) and sterilized at 250° F. for 15 minutes. After the autoclave the samples are left to cool down to room temperature and then placed in refrigerator. The samples are homogenized by mixing the vials while cold.

Appearance (e.g., discoloration and/or precipitation) is observed and recorded. HPLC analysis is performed using an Agilent 1200 equipped with a Luna C18(2) 3 μm, 1001, 250×4.6 mm column) using a 30-80 acetonitrile gradient (1-10 min) of (water-acetonitrile mixture containing 0.05% TFA), for a total run of 15 minutes. Samples are diluted by taking 304 of sample and dissolved with 1.5 mL of a 1:1 acetonitrile water mixture. Purity of the otic agent in the autoclaved samples is recorded.

Formulations comprising the otic agents and/or the EAC protectants, prepared according to the procedure above, are tested using the above procedure to determine the effect of pH on degradation during the autoclaving step.

### Example 18—Effect of Buffer Type on the Degradation Products for Formulations Containing Poloxamer 407 after Heat Sterilization (Autoclaving)

A TRIS buffer is made by dissolving 377.8 mg of sodium chloride (Fisher Scientific), and 602.9 mg of Tromethamine (Sigma Chemical Co.) then QS to 100 g with sterile filtered DI water, pH is adjusted to 7.4 with 1M HCl.

### Stock Solution Containing 25% Poloxamer 407 Solution in TRIS Buffer:

Weigh 45 g of TRIS buffer, chill in an ice chilled bath then sprinkle into the buffer, while mixing, 15 g of poloxamer 407 (Spectrum Chemicals). The mixture is further mixed until all the poloxamer is completely dissolved.

A series of formulations is prepared with the above stock solution. An appropriate amount of otic agent (or salt or prodrug thereof) and/or otic agent as micronized/coated/liposomal particles (or salt or prodrug thereof) is used for all experiments.

### Stock Solution (pH 7.3) Containing 25% Poloxamer 407 Solution in PBS Buffer:

PBS buffer described above is used. Dissolve 704 mg of sodium chloride (Fisher Scientific), 601.2 mg of sodium phosphate dibasic anhydrous (Fisher Scientific), 242.7 mg of sodium phosphate monobasic anhydrous (Fisher Scientific) with 140.4 g of sterile filtered DI water. The solution is cooled down in an ice chilled water bath and then 50 g of poloxamer 407 is sprinkled into the cold solution while mixing. The mixture is further mixed until the poloxamer is completely dissolved.

A series of formulations is prepared with the above stock solution. An appropriate amount of otic agent (or salt or prodrug thereof) and/or otic agent as micronized/coated/liposomal particles (or salt or prodrug thereof) is used for all experiments.

Tables 13 and 14 list samples prepared using the procedures described above. An appropriate amount of otic agent is added to each sample to provide a final concentration of 2% otic agent in the sample.

TABLE 13
Preparation of samples containing TRIS buffer

| | | 25% Stock | TRIS |
|---|---|---|---|
| Sample | pH | Solution (g) | Buffer (g) |
| 20% P407/2% otic agent/TRIS | 7.45 | 8.01 | 1.82 |
| 18% P407/2% otic agent/TRIS | 7.45 | 7.22 | 2.61 |
| 16% P407/2% otic agent/TRIS | 7.45 | 6.47 | 3.42 |
| 18% P407/2% otic agent/TRIS | 7.4 | 7.18 | 2.64 |
| 4% otic agent/TRIS | 7.5 | — | 9.7 |
| 2% otic agent/TRIS | 7.43 | — | 5 |
| 1% otic agent/TRIS | 7.35 | — | 5 |
| 2% otic agent/TRIS | 7.4 | — | 4.9 |
| (suspension) | | | |

TABLE 14
Preparation of samples containing PBS buffer (pH of 7.3)

| | 25% Stock Solution | |
|---|---|---|
| Sample | in PBS (g) | PBS Buffer (g) |
| 20% P407/2% otic agent/PBS | 8.03 | 1.82 |
| 18% P407/2% otic agent/PBS | 7.1 | 2.63 |
| 16% P407/2% otic agent/PBS | 6.45 | 3.44 |
| 18% P407/2% otic agent/PBS | — | 2.63 |
| 2% otic agent/PBS | — | 4.9 |

One mL samples are individually placed in 3 mL screw cap glass vials (with rubber lining) and closed tightly. The vials are placed in a Market Forge-sterilmatic autoclave (setting, slow liquids) and sterilized at 250° F. for 25 minutes. After the autoclaving the samples are left to cool down to room temperature. The vials are placed in the refrigerator and mixed while cold to homogenize the samples.

HPLC analysis is performed using an Agilent 1200 equipped with a Luna C18(2) 3 μm, 1001, 250×4.6 mm column) using a 30-80 acetonitrile gradient (1-10 min) of (water-acetonitrile mixture containing 0.05% TFA), for a total run of 15 minutes. Samples are diluted by taking 30 μL of sample and dissolving with 1.5 mL of a 1:1 acetonitrile water mixture. Purity of the otic agent in the autoclaved samples is recorded. The stability of formulations in TRIS and PBS buffers is compared.

Viscosity measurements are performed using a Brookfield viscometer RVDV-II+P with a CPE-51 spindle rotated at 0.08 rpm (shear rate of 0.31 s⁻¹), equipped with a water jacketed temperature control unit (temperature ramped from 15-34° C. at 1.6° C./min). Tgel is defined as the inflection point of the curve where the increase in viscosity occurs due to the sol-gel transition. Only formulations that show no change after autoclaving are analyzed.

Formulations comprising the otic agents and/or the EAC protectants, prepared according to the procedures described herein, are tested using the above procedure to determine the effect addition of a secondary polymer on the degradation products and viscosity of a formulation containing 2% active agent and 17% poloxamer 407 after heat sterilization (autoclaving). Stability of formulations containing micronized otic agent is compared to non-micronized otic agent formulation counterparts.

### Example 19—In Vitro Comparison of Release Profile

Dissolution is performed at 37° C. in snapwells (6.5 mm diameter polycarbonate membrane with a pore size of 0.4 μm), 0.2 mL of a gel formulation described herein is placed into snapwell and left to harden, then 0.5 mL buffer is placed into reservoir and shaken using a Labline orbit shaker at 70 rpm. Samples are taken every hour (0.1 mL withdrawn and replace with warm buffer). Samples are analyzed for otic agent concentration by UV at 245 nm against an external calibration standard curve. Pluronic concentration is analyzed at 624 nm using the cobalt thiocyanate method. Relative rank-order of mean dissolution time (MDT) as a function of % P407 is determined. A linear relationship between the formulations mean dissolution time (MDT) and the P407 concentration indicates that the otic agent is released due to the erosion of the polymer gel (poloxamer) and not via diffusion. A non-linear relationship indicates release of otic agent via a combination of diffusion and/or polymer gel degradation.

Alternatively, samples are analyzed using the method described by Li Xin-Yu paper [Acta Pharmaceutica Sinica 2008,43(2):208-203] and Rank-order of mean dissolution time (MDT) as a function of % P407 is determined.

Formulations comprising the otic agents and/or the EAC protectants, prepared according to the procedures described herein, are tested using the above procedure to determine the release profile of the otic agents.

### Example 20—Determination of Temperature Range for Sterile Filtration

The viscosity at low temperatures is measured to help guide the temperature range at which the sterile filtration needs to occur to reduce the possibility of clogging.

Viscosity measurements are performed using a Brookfield viscometer RVDV-II+P with a CPE-40 spindle rotated at 1, 5 and 10 rpm (shear rate of 7.5, 37.5 and 75 s⁻¹), equipped with a water jacketed temperature control unit (temperature ramped from 10-25° C. at 1.6° C./min).

The Tgel of a 16% Pluronic P407 is determined as a function of increasing concentration of otic agent. The increase in Tgel for a 16% pluronic formulation is estimated by:

ΔT_{gel}=0.93[% otic agent]

Formulations comprising the otic agents and/or the EAC protectants, prepared according to procedures described herein, are tested using the above procedure to determine the temperature range for sterile filtration. The effect of addition of increased amounts of otic agent on the Tgel, and the apparent viscosity of the formulations is recorded.

### Example 21—Determination of Manufacturing Conditions

TABLE 17
Viscosity of potential formulations at manufacturing/filtration conditions.

| | Apparent Viscosity^{a} (cP) | | |
|---|---|---|---|
| Sample | 5° C. below Tgel | 20° C. | Temperature @ 100 cP |
| Placebo | 52 cP @ 17° C. | 120 cP | 19° C. |
| 16% P407/2% | 90 cP @ 18° C. | 147 cP | 18.5° C. |
| otic agent | | | |
| 16% P407/6% | 142 cP @ 22° C. | 105 cP | 19.7° C. |
| otic agent | | | |

^{a}Viscosity measured at a shear rate of 37.5 s⁻¹

An 8 liter batch of a 16% P407 placebo is manufactured to evaluate the manufacturing/filtration conditions. The placebo is manufactured by placing 6.4 liters of DI water in a 3 gallon SS pressure vessel, and left to cool down in the refrigerator overnight. The following morning the tank is taken out (water temperature 5° C., RT 18° C.) and 48 g of sodium chloride, 29.6 g of sodium phosphate dibasic dehydrate and 10 g of sodium phosphate monobasic monohydrate is added and dissolved with an overhead mixer (IKA RW20 @ 1720 rpm). Half hour later, once the buffer is dissolved (solution temperature 8° C., RT 18° C.), 1.36 kg of poloxamer 407 is slowly sprinkled into the buffer solution in a 15 minute interval (solution temperature 12° C., RT 18° C.), then speed is increased to 2430 rpm. After an additional one hour mixing, mixing speed is reduced to 1062 rpm (complete dissolution).

The temperature of the room is maintained below 25° C. to retain the temperature of the solution at below 19° C. The temperature of the solution is maintained at below 19° C. up to 3 hours of the initiation of the manufacturing, without the need to chill/cool the container.

Three different Sartoscale (Sartorius Stedim) filters with a surface area of 17.3 cm² are evaluated at 20 psi and 14° C. of solution

1) Sartopore 2, 0.2 μm 5445307HS-FF (PES), flow rate of 16 mL/min

2) Sartobran P, 0.2 μm 5235307HS-FF (cellulose ester), flow rate of 12 mL/min

3) Sartopore 2 XLI, 0.2 μm 5445307IS-FF (PES), flow rate of 15 mL/min

Sartopore 2 filter 5441307H4-SS is used, filtration is carried out at the solution temperature using a 0.45, 0.2 μm Sartopore 2 150 sterile capsule (Sartorius Stedim) with a surface area of 0.015 m² at a pressure of 16 psi. Flow rate is measured at approximately 100 mL/min at 16 psi, with no change in flow rate while the temperature is maintained in the 6.5-14° C. range. Decreasing pressure and increasing temperature of the solution causes a decrease in flow rate due to an increase in the viscosity of the solution. Discoloration of the solution is monitored during the process.

TABLE 18
Predicted filtration time for a 16% poloxamer 407 placebo at a solution
temperature range of 6.5-14° C. using Sartopore 2, 0.2 μm filters
at a pressure of 16 psi of pressure.

| | | Estimated flow rate | Time to filter 8 L |
|---|---|---|---|
| Filter | Size (m²) | (mL/min) | (estimated) |
| Sartopore 2, size 4 | 0.015 | 100 mL/min | 80 min |
| Sartopore 2, size 7 | 0.05 | 330 mL/min | 24 min |
| Sartopore 2, size 8 | 0.1 | 670 mL/min | 12 min |

Viscosity, Tgel and UV/Vis absorption is checked before filtration evaluation. Pluronic UV/Vis spectra are obtained by a Evolution 160 UV/Vis (Thermo Scientific). A peak in the range of 250-300 nm is attributed to BHT stabilizer present in the raw material (poloxamer). Table 19 lists physicochemical properties of the above solutions before and after filtration.

TABLE 19
Physicochemical properties of 16% poloxamer 407 placebo solution before
and after filtration

| | Tgel | Viscosity^{a} @ 19° C. | |
|---|---|---|---|
| Sample | (° C.) | (cP) | Absorbance @ 274 nm |
| Before filtration | 22 | 100 | 0.3181 |
| After filtration | 22 | 100 | 0.3081 |

^{a}Viscosity measured at a shear rate of 37.5 s⁻¹

The above process is applicable for manufacture of 16% P407 formulations, and includes temperature analysis of the room conditions. Preferably, a maximum temperature of 19° C. reduces cost of cooling the container during manufacturing. In some instances, a jacketed container is used to further control the temperature of the solution to ease manufacturing concerns.

### Example 22—In Vitro Release of Otic Agent from an Autoclaved Micronized Sample

16% poloxamer 407/1.5% otic agent in TRIS buffer: 250.8 mg of sodium chloride (Fisher Scientific), and 302.4 mg of Tromethamine (Sigma Chemical Co.) is dissolved in 39.3 g of sterile filtered DI water, pH is adjusted to 7.4 with 1M HCl. 4.9 g of the above solution is used and an appropriate amount of micronized otic agent is suspended and dispersed well. 2 mL of the formulation is transferred into a 2 mL glass vial (Wheaton serum glass vial) and sealed with 13 mm butyl styrene (kimble stoppers) and crimped with a 13 mm aluminum seal. The vial is placed in a Market Forge-sterilmatic autoclave (settings, slow liquids) and sterilized at 250° F. for 25 minutes. After the autoclaving the sample is left to cool down to room temperature. The vial is placed in the refrigerator and mixed while cold to homogenize the sample. Sample discoloration or precipitation after autoclaving is recorded.

Dissolution is performed at 37° C. in snapwells (6.5 mm diameter polycarbonate membrane with a pore size of 0.4 μm), 0.2 mL of gel is placed into snapwell and left to harden, then 0.5 mL PBS buffer is placed into reservoir and shaken using a Labline orbit shaker at 70 rpm. Samples are taken every hour [0.1 mL withdrawn and replaced with warm PBS buffer containing 2% PEG-40 hydrogenated castor oil (BASF) to enhance otic agent solubility]. Samples are analyzed for otic agent concentration by UV at 245 nm against an external calibration standard curve. The release rate is compared to other formulations disclosed herein. MDT time is calculated for each sample.

Solubilization of otic agent in the 16% poloxamer system is evaluated by measuring the concentration of the otic agent in the supernatant after centrifuging samples at 15,000 rpm for 10 minutes using an eppendorf centrifuge 5424. Otic agent concentration in the supernatant is measured by UV at 245 nm against an external calibration standard curve.

Formulations comprising the otic agents and/or the EAC protectants, prepared according to the procedures described herein, are tested using the above procedures to determine release rate of the otic agent from each formulation.

### Example 23—Effect of Poloxamer Concentration and Otic Agent Concentration on Release Kinetics

A series of compositions comprising varying concentrations of a gelling agent and micronized otic agent is prepared using procedures described above. The mean dissolution time (MDT) for each composition in Table 20 is determined using procedures described above.

TABLE 20
Preparation of poloxamer/otic agent compositions

| Sample | pH |
|---|---|
| 15.5% P407/1.5% otic agent/PBS | 7.4 |
| 16% P407/1.5% otic agent/PBS | 7.4 |
| 17% P407/1.5% otic agent/PBS | 7.4 |
| 15.5% P407/4.5% otic agent/PBS | 7.4 |
| 16% P407/4.5% otic agent/PBS | 7.4 |
| 17% P407/4.5% otic agent/PBS | 7.4 |

The effect of gel strength and otic agent concentration on release kinetics of an otic agent from the composition is determined by measurement of the MDT for poloxamer, and measurement of MDT for otic agent.

The apparent viscosity of each composition is measured as described above. A thermoreversible polymer gel concentration of about 15.5% in a composition described above provides an apparent viscosity of about 270,000 cP. A thermoreversible polymer gel concentration of about 16% in a composition described above provides an apparent viscosity of about 360,000 cP. A thermoreversible polymer gel concentration of about 16% in a composition described above provides an apparent viscosity of about 480,000 cP.

Compositions comprising the otic agents and/or the EAC protectants, prepared according to the procedures described above are tested using the above procedure to determine release rate of the otic agent from each composition.

### Example 24—In Vivo Testing of Otic Agent Formulation in Guinea Pigs

A cohort of guinea pigs (Charles River, females weighing 200-300 g) is injected with 50 μL of different P407-otic agent formulations described herein, containing 0 to 50% otic agent. The gel elimination time course for each formulation is determined. A faster gel elimination time course of a formulation indicates lower mean residence time (MRT). Thus the injection volume and the concentration of an otic agent in a formulation are tested to determine optimal parameters for preclinical and clinical studies.

### Example 25—In Vivo Extended Release Kinetics

A cohort of 21 guinea pigs (Charles River, females weighing 200-300 g) is injected with 50 μL 16% P407 formulation buffered at 280 mOsm/kg and containing 0.1% to 35% otic agent by weight of the formulation. Animals are dosed on day 1. The release profile for the formulations is determined based on analysis of the EAC.

### Example 26—Clinical Trials of Otic Formulations in Ceruminosis Patients Study Objective

The primary objective of this study will be to assess the safety and efficacy of the otic formulations disclosed herein compared with that of a placebo to ameliorate ceruminosis symptoms in afflicted patients.

Methods

Study Design

This will be a phase 3, multicentre, double-blind, randomised, placebo-controlled, three-arm study comparing an otic formulation disclosed herein (100 mg and 200 mg) to placebo in the treatment of ceruminosis symptoms. Approximately 150 subjects will be enrolled in this study, and randomised (1:1) to 1 of 3 treatment groups based on a randomization sequence prepared by sponsor. Each group will receive 200 mg controlled release otic formulation, 400 mg controlled release otic formulation, or controlled release placebo formulation.

After a 1-week baseline phase, patients from each group will be randomized to a 16 week double treatment period (8-week treatment followed by an 8-week maintenance period). Primary efficacy will be measured as a percentage change in the frequency and intensity of ceruminosis symptoms, including dizziness, loss of hearing, tinnitus, and incidence of earache after treatment as compared to baseline measurements. Further, visual inspection of the EAC using standard examination procedures accompanies each measurement.

While preferred embodiments of the present invention have been shown and described herein, such embodiments are provided by way of example only. Various alternatives to the embodiments described herein are optionally employed in practicing the inventions. It is intended that the following claims define the scope of the invention and that methods and structures within the scope of these claims and their equivalents be covered thereby.

We claim:

1. A pharmaceutical composition comprising an otic agent for modulating the production of cerumen; and an auris-acceptable gel.

2. The pharmaceutical composition of claim 1, wherein the auris-acceptable gel is an aqueous auris-acceptable gel.

3. The pharmaceutical composition of any one of claims 1-2, wherein the auris-acceptable gel is an auris external-acceptable gel.

4. The pharmaceutical composition of claim 3, wherein the auris external-acceptable gel is an auris-acceptable thermoreversible gel.

5. The pharmaceutical composition of any one of claims 1-4, wherein the composition has a gelation temperature between about 19° C. to about 42° C.

6. The pharmaceutical composition of any one of claims 1-5, wherein the composition has an apparent viscosity of about 15,000 cP to about 1,000,000 cP.

7. The pharmaceutical composition of any one of claims 1-5, wherein the composition has an apparent viscosity of about 100,000 cP to about 500,000 cP.

8. The pharmaceutical composition of any one of claims 1-5, wherein the composition has an apparent viscosity of about 250,000 cP to about 500,000 cP.

9. The pharmaceutical composition of any one of claims 1-8, wherein the composition has a practical osmolarity between about 150 to about 500 mOsm/L.

10. The pharmaceutical composition of any one of claims 1-8, wherein the composition has a practical osmolarity between about 200 to about 400 mOsm/L.

11. The pharmaceutical composition of any one of claims 1-8, wherein the composition has a practical osmolarity between about 250 to about 320 mOsm/L.

12. The pharmaceutical composition of any one of claims 1-11, wherein the otic agent has a mean dissolution time of about 30 hours.

13. The pharmaceutical composition of any one of claims 1-12, wherein the otic agent is released from the composition over a period of at least 3 days.

14. The pharmaceutical composition of any one of claims 1-12, wherein the otic agent is released from the composition over a period of at least 4 days.

15. The pharmaceutical composition of any one of claims 1-12, wherein the otic agent is released from the composition over a period of at least 5 days.

16. The pharmaceutical composition of any one of claims 1-12, wherein the otic agent is released from the composition over a period of at least 7 days.

17. The pharmaceutical composition of any one of claims 1-12, wherein the otic agent is released from the composition over a period of at least 14 days.

18. The pharmaceutical composition of any one of claims 1-17, wherein the otic agent is in the form of a neutral molecule, free acid, free base, a salt, a prodrug, or a combination thereof.

19. The pharmaceutical composition of any one of claims 1-18, wherein the otic agent comprises multiparticulates.

20. The pharmaceutical composition of any one of claims 1-19, wherein the otic agent is essentially in the form of micronized particles.

21. The pharmaceutical composition of any one of claims 1-19, wherein the otic agent is in the form of micronized particles.

22. The pharmaceutical composition of any one of claims 1-21, wherein the pH of the composition is between about 5.5 to about 9.0.

23. The pharmaceutical composition of any one of claims 1-21, wherein the pH of the composition is between about 6.0 to about 8.5.

24. The pharmaceutical composition of any one of claims 1-21, wherein the pH of the composition is between about 7.0 to about 8.0.

25. The pharmaceutical composition of any one of claims 1-24, wherein the composition is essentially free of alcohol solvent.

26. The pharmaceutical composition of any one of claims 1-24, wherein the composition is essentially free of glycol solvent.

27. The pharmaceutical composition of any one of claims 1-26, wherein the auris-acceptable gel is bioerodable.

28. The pharmaceutical composition of any one of claims 1-27, wherein the otic agent is choline ester or carbamate, plant alkaloid, reversible cholinesterase inhibitor, acetylcholine release promoter, anti-adrenergy, sympathomimetic, or a combination thereof.

29. The pharmaceutical composition of claim 28, wherein the otic agent is choline ester or carbamate, preferrably acetylcholine or carbachol.

30. The pharmaceutical composition of claim 28, wherein the otic agent is plant alkaloid, preferably pilocarpine.

31. The pharmaceutical composition of claim 28, wherein the otic agent is reversible cholinesterase inhibitor, preferably neostigmine or physostigmine.

32. The pharmaceutical composition of claim 28, wherein the otic agent is acetylcholine release promoter, preferably droperidol, resperidone, or trazodone.

33. The pharmaceutical composition of claim 28, wherein the otic agent is anti-adrenergic, preferably clonidine, propranolol, atenolol, or prazosin.

34. The pharmaceutical composition of claim 28, wherein the otic agent is sympathomimetic, preferably norepinephrine, or dopamine.

35. The pharmaceutical composition of any one of claims 1-34, wherein the composition comprises about 0.1% to about 20% by weight of the otic agent.

36. The pharmaceutical composition of any one of claims 1-34, wherein the composition comprises about 1% to about 10% by weight of the otic agent.

37. The pharmaceutical composition of any one of claims 1-34, wherein the composition comprises about 5% to about 8% by weight of the otic agent.

38. The pharmaceutical composition of any one of claims 1-37, wherein the composition further comprises one or more EAC protectant.

39. The pharmaceutical composition of claim 38, wherein the EAC protectant is selected from squalene, lanosterol, and cholesterol.

40. The pharmaceutical composition of claim 38, wherein the EAC protectant is one or more antimicrobial agent.

41. The pharmaceutical composition of claim 40, wherein the antimicrobial agent is an antimicrobial peptide.

42. The pharmaceutical composition of any one of claims 1-41, wherein the composition is used in the treatment of ceruminosis.

43. The pharmaceutical composition of claim 42, wherein ceruminosis is associated with a disease or condition.

44. The pharmaceutical composition of claim 43, wherein the disease or condition is ear pruritus, otitis externa, otalgia, tinnitus, vertigo, ear fullness, hearing loss, or a combination thereof.

45. A method of modulating cerumen production comprising administering to an individual in need thereof a pharmaceutical composition comprising an amount of an otic agent that modulates cerumen production; and an auris-acceptable gel.

46. A method of treating cerumenosis comprising administering to an individual in need thereof a pharmaceutical composition comprising an amount of an otic agent that modulates cerumen production; and an auris-acceptable gel.

47. The method of claim 46, wherein ceruminosis is associated with a disease or condition.

48. The method of claim 47, wherein the disease or condition is ear pruritus, otitis externa, otalgia, tinnitus, vertigo, ear fullness, hearing loss, or a combination thereof.

49. The method of any one of claims 45-48, wherein the composition is administered locally to the external auditory canal, the outer surface of the tympanic membrane, or a combination thereof.

50. The method of any one of claims 45-49, wherein the composition is not administered through the tympanic membrane.

51. The method of any one of claims 45-50, further comprising administering an EAC protectant to the individual in need thereof.

52. The method of claim 51, wherein the EAC protectant is selected from squalene, lanosterol, and cholesterol.

53. The method of claim 51, wherein the EAC protectant is one or more antimicrobial agent.

54. The method of claim 53, wherein the antimicrobial agent is an antimicrobial peptide.

55. The method of any one of claims 51-54, wherein the EAC protectant is incorporated into the pharmaceutical composition comprising the otic agent.

56. The method of any one of claims 51-54, wherein the EAC protectant is formulated into a supplemental composition administered separately from the pharmaceutical composition comprising the otic agent.

57. The method of claim 56, wherein the supplemental composition further comprises an auris-acceptable gel.

58. The method of claim 56 or claim 57, wherein the supplemental composition is administered locally to the external auditory canal, the outer surface of the tympanic membrane, or a combination thereof.

59. The method of any one of claims 56-58, wherein the supplemental composition is not administered through the tympanic membrane.

60. The method of any one of claims 45-59, wherein the pharmaceutical composition is according to any one of claims 1-44.

61. The method of any one of claims 45-60, wherein the pharmaceutical composition does not provide sustained release of the otic agent that modulates cerumen production into the middle ear or inner ear.

62. The method of any one of claims 45-61, wherein the pharmaceutical composition does not provide any release of the otic agent that modulates cerumen production into the middle ear or inner ear.

63. The pharmaceutical composition of any one of claims 1-44, wherein the pharmaceutical composition does not provide sustained release of the otic agent that modulates cerumen production into the middle ear or inner ear.

64. The pharmaceutical composition of any one of claims 1-44 and 63, wherein the pharmaceutical composition does not provide any release of the otic agent that modulates cerumen production into the middle ear or inner ear.
